(19) United States
(12) Patent Application Publication
(10) Pub. No.: US 2022/0168472 A1
(43) Pub. Date: Jun. 2, 2022

(54) ENZYME-DEGRADABLE HYDROGEL FOR DELIVERY OF A PAYLOAD

(71) Applicants: Susmita BOSE, Waterloo (CA); John TSE, Waterloo (CA); Muhammad RIZWAN, North York (CA); Evelyn YIM, Waterloo (CA); Lyndon JONES, Waterloo (CA); Chau-Minh PHAN, Waterloo (CA)

(72) Inventors: Susmita Bose, Waterloo (CA); John Tse, Waterloo (CA); Muhammad Rizwan, North York (CA); Evelyn Yim, Waterloo (CA); Lyndon Jones, Waterloo (CA); Chau-Minh Phan, Waterloo (CA)

(21) Appl. No.: 17/438,799

(22) PCT Filed: Mar. 13, 2020
(86) PCT No.: PCT/CA2020/050345

§ 371 Date: Sep. 13, 2021

Related U.S. Application Data
(60) Provisional application No. 62/919,425, filed on Mar. 13, 2019.

(51) Int. Cl. A61L 27/54; A61K 9/06; A61K 47/42; A61K 38/48; A61K 38/40; A61K 31/728; A61K 38/18; A61K 38/28; A61P 17/02; A61L 27/22; A61L 15/32; A61L 15/60

(52) U.S. Cl. CPC A61L 27/54; A61F 9/0017; A61K 47/42; A61K 38/4886; A61K 38/40; A61K 31/728; A61K 38/1808; A61K 38/28; A61K 38/1858; A61K 38/1841; A61K 38/18; A61K 38/1833; A61K 38/1825; A61P 17/02; A61L 27/222; A61L 15/32; A61L 15/60; A61L 2430/16; A61K 9/06

(57) ABSTRACT

Various embodiments are described herein for the fabrication enzyme degradable hydrogels useful as payload delivery systems. More particularly, embodiments disclosed herein relate to enzyme-degradable hydrogel systems comprising a crosslinkable polymer, such as a chemically-modified biopolymer, for example, chemically-modified gelatin, the hydrogel formed by a method comprising sequential physical and chemical crosslinking steps, for delivery of various payloads. Enzymes may be selected and administered to tune the release profile of the hydrogel. The payload can be, but not limited to, drugs, markers, cells, or these members encapsulated within another drug delivery such as a nanoparticle, or liposome. The hydrogel system can also be combined with another device such as a contact lens or bandage for wound healing.

### FIELD

Disclosed herein is an enzyme-degradable hydrogel delivery system useful for delivery of a variety of payloads, as well as devices and methods related thereto. The hydrogel is formed by a method comprising sequential physical and chemical crosslinking steps. The properties and release profile of the hydrogel can be tuned as described herein. Enzymes that facilitate hyrodgel degradation can be selected and administered to further tune the system.

### BACKGROUND

Delivery systems are used to protect and/or carry payloads, such as drugs and other active compounds, for a length of time or until they reach a target site where they can be released to achieve a desired effect. The majority of drug delivery systems rely on passive diffusion to gradually release their payloads. Such passive delivery systems are not truly controlled delivery systems, since the quantity and timing of drug release is not controlled on demand.

Gelatin-based hydrogels, such as methacrylated gelatin (GelMA) hydrogels, are enzyme-degradable hydrogels derived from collagen. GelMA hydrogels have been studied extensively in tissue engineering applications, such as tissue scaffolding, but are not generally considered for drug delivery applications due to their poor mechanical strength and high porosity. For example, given their use as soft biocompatible gels for cell encapsulation, or as cell scaffolds, they are necessarily freely diffusive such that nutrients and large molecules can diffuse to and from the cells.

The present inventors previously disclosed a method of manufacturing a GelMA hydrogel having improved characteristics for tissue engineering applications (GelMA+), such as an over 8-fold increase in mechanical strength compared to regular GelMA, and favorable biodegradation kinetics both in vitro and in vivo. The method involved sequential crosslinking steps, wherein a solution of methacrylated gelatin was incubated at a cool temperature for a sufficient amount to permit physical association and crosslinking prior to UV crosslinking (see Rizwan et al. Sequentially-Crosslinked Bioactive Hydrogels as Nano-Patterned Substrates with Customizable Stiffness and Degradation for Corneal Tissue Engineering Applications. Biomaterials 2017 v.120: pp. 139-54, the entire contents of which are incorporated herein by reference). Even though the hydrogel had improved characteristics for tissue engineering applications, it was not known whether such a hydrogel could be useful for drug delivery applications given the general drawbacks of hydrogels for such applications.

### SUMMARY OF VARIOUS EMBODIMENTS

In one aspect, the invention provides a hydrogel system for delivering a payload comprising: a hydrogel comprising a crosslinkable polymer, the hydrogel formed by a method comprising sequential physical and chemical crosslinking steps; and a payload.

In some embodiments, the polymer is a chemically-modified biopolymer. In some chemically-modified biopolymer comprises chemically-modified gelatin, such as gelatin methacrylate (GelMA). In some embodiments, the hydrogel system is degradable by an enzyme, such as a matrix metalloproteinase, collagenase, or gelatenase. In some embodiments, the physical crosslinking step comprises incubating a solution comprising the chemically-modified gelatin under suitable conditions and for a sufficient period of time to permit physical crosslinking of at least a portion of the chemically-modified gelatin. In some embodiments, the chemical crosslinking step is a thermal or photochemical process between functional groups on the chemically-modified gelatin.

In one aspect, the invention provides a device for delivering a payload comprising the hydrogel system according to embodiments herein. In some embodiments, the device is a lens, implant, insert or wound dressing, and the device may use the hydrogel system to release the payload over a release period.

In another aspect, the invention provides a method of making a hydrogel system for delivering a payload, the method comprising: providing a crosslinkable polymer (such as a chemically-modified biopolymer, for example, gelatin); physical crosslinking of the polymer; chemical crosslinking of the polymer; and introducing a payload into the hydrogel so-formed, which hydrogel may be the hydrogel system according to any of the embodiments of the present disclosure.

The present disclosure also provides methods of making devices for delivering a payload comprising the hydrogel system disclosed herein. Also provided are pharmaceutical compositions, and methods for delivering payloads to a patient and treating wounds, using the hydrogel system of the present disclosure.

Other features and advantages of the present application will become apparent from the following detailed description, taken together with the accompanying drawings. It should be understood, however, that the detailed description and the specific examples, while indicating preferred embodiments of the application, are given by way of illustration only, since various changes and modifications within the spirit and scope of the application will become apparent to those skilled in the art from this detailed description.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described.

FIG. 1 shows an example of a GelMA+ drug delivery system consisting of GelMA+ (GELMA 100), a payload (PAYLOAD 100), and an enzyme trigger (ENZYME 100).

FIG. 2 Shows an example of a process to make GelMA+ (GELMA+ 200) involving physical and chemical cross-linking steps.

FIG. 3 shows an example of a payload. The payload may be a drug (PAYLOAD 301), a drug encapsulated in a nanoparticle (PAYLOAD 302), or a drug covalently linked to the GelMA+ (PAYLOAD 303).

FIG. 4 shows a mechanism for an enzyme-triggered degradation of GelMA+.

FIG. 5 shows an example of an enzyme-triggered release of a payload from GelMA+.

FIG. 6 shows an example application of a GelMA+ system for wound healing.

FIG. 7 shows an example application of a GelMA+ system used in combination with another device, such as a contact lens (DEVICE 700) for delivering a payload for use in corneal wound healing.

FIG. 8 shows an example application of a GelMA+ system used for delivering a payload for use in wound healing, where the addition of the enzyme trigger is from an external source.

FIG. 9 shows percent release of FITC-Dextran from GelMA+ after 24 hours (n=3), as outlined in Example 1.

FIG. 10 shows release of FITC-dextran from GelMA+ in response to increasing MMP9 concentration after 24 hours (n=3), as outlined in Example 1.

FIG. 11 shows release of FITC-Dextran from 10% (w/v) GelMA+ formulation in varying concentrations of MMP-9 over 24 hours, as outlined in Example 2.

FIG. 12 shows release of FITC-Dextran from 20% (w/v) GelMA+ formulation in varying concentrations of MMP-9 over 24 hours, as outlined in Example 2.

FIG. 13 shows release of FITC-Dextran from 30% (w/v) GelMA+ formulation in varying concentrations of MMP-9 over 24 hours, as outlined in Example 2.

FIG. 14 shows release of FITC-Dextran from varying GelMA+ formulations in varying concentrations of MMP-9 at 24 hours, as outlined in Example 2.

FIG. 15 shows release of bovine lactoferrin from 30% (w/v) GelMA+ formulation in varying concentrations of MMP-9 over 24 hours (n=4), as outlined in Example 2.

FIG. 16 shows A. A schematic representing the degradation of gelatin methacrylate (GelMA+) hydrogels loaded with hyaluronic acid (HA) via matrix metalloproteinase (MMP) enzymes. The MMP enzymes cleave the gelatin compounds, releasing HA to local corneal epithelial cells (CEpCs). HA is known to promote cell migration and wound healing, and the controlled release of HA over an extended period of time offers improved CEpC regeneration. B. A table representing the different variation of GelMA and GelMA+ hydrogels. Three variables during the fabrication process were investigated: gelatin concentration, methacrylation degree, and physical crosslinking, as outlined in Example 3.

FIG. 17 shows the effect of molecular weight of loaded drug on GelMA and GelMA+ controlled release profile. To observe small molecular weight, 4 kDa Fluorescein isothiocyanate (FITC)-dextran was loaded into the various GelMA− and GelMA+ hydrogels and incubated in A. phosphate-buffered saline (PBS) or B. 1 μg/mL MMP-8 solution (n=4). Similarly, a larger molecular weight of 70 kDa FITC-dextran was loaded into the various GelMA and GelMA+ hydrogels and incubated in C. PBS or D. 1 μg/mL MMP-8 solution (n=4). Error bars shown are SD, as outlined in Example 3.

FIG. 18 shows release profiles of the various GelMA and GelMA+ hydrogel samples with varying concentration (10%, 20%, or 30%), crosslinking steps (− or +) and varying methacrylation degree. GelMA/GelMA+ samples were incubated with 1 μg/ml matrix metalloproteinase (MMP)-8 enzyme and the release of FITC-dextran (70 kDa) was recorded for 7 days. Error bars shown are standard deviation (SD). The A. low degree of methacrylation (L) were fully degraded within three days. It is particularly evident that within the B. first 12 hours a burst release occurred, and a reduced rate of release followed. The C. high degree of methacrylation showed a slower release characteristic compared to the low degree of methacrylation and an overall longer degradation time. From D. the 12 hour inset of high degrees of methacrylation GelMA/GelMA+, there was a significantly reduced burst release of FITC-Dextran, as outlined in Example 3.

FIG. 19 shows release profile of A. high degrees of methacrylation GelMA/GelMA+ hydrogels degraded with 250 ng/ml MMP-9. The released FITC-dextran (70 kDa) was collected to determine the amount released by the GelMA/GelMA+ (n=19). Error bars shown are SD. However, MMP-9 did not reduce the burst release of FITC-dextran from the GelMA/GelMA+ hydrogels as seen in B. the first 12 hours of degradation, as outlined in Example 3.

FIG. 20 shows the effect of bolus dosages of 500 kDa HA upon PDMS wound assays of A. RCEPCs (n=4) and B. HCEpCs (n=5). Error bars shown are SD. The RCEpCs showed improved healing with both 0.75 and 0.45 mg/ml of HA. However, 0.45 mg/ml HA offered a greater wound healing effect compared to the higher concentration. This effect was reversed in the HCEpC assay where the higher concentration of HA showed a greater improvement compared to the lower concentration. It is also noted that the time to wound closure for RCEpC was significantly faster than the closure time of HCEpCs at 2 days and 7 days. The progression of the wound from C. 0, 48, and 96 hours were recorded using microscope imaging and analyzed with Graph Pad Prism, as outlined in Example 3.

FIG. 21 shows A. The efficacy of 60 kDa HA bolus dosages on wound healing was investigated in a PDMS wound assay (n=5). Error bars shown are SD. The resulting data showed a therapeutic range of 0.1-0.6 mg/ml for 60 kDa HA. The administration of MMP-9 to a wound assay resulted in a detrimental effect to the wound healing process. B. Data table of the wound closure percentages of daily treatments of 60 kDa HA to CEpC PDMS stencil wound assays. It is noted the therapeutic window of 60 kDa HA is from 0.1 mg/ml to 0.6 mg/ml, as outlined in Example 3.

FIG. 22 shows A. Controlled release of 60 kDa HA from 10H− GelMA on 1 mm HCEpCs PDMS wound assay (n=4) was conducted to verify the bioactivity of the HA loaded hydrogels. All GelMA samples degraded in MMP-9 showed improved wound healing compared to the control, including the 10H− sample not loaded with HA. B. ELISA of 60 kDa HA released from 10H− GelMA (n=2). The estimated burst release and release rate over seven days was 85% and 1.5%. The actual burst release for 750, 550, 250, and 150 μg was 83%, 62%, 49%, and 68% respectively. The 150, 250, and 550 μg samples exhibited a 5% daily release rate and the 750 μg samples a 2% release rate, as outlined in Example 3.

FIG. 23 shows MMP-8 VS MMP-9 enzyme on 20H− GelMA patch (n=9). Error bars shown are SD, as outlined in Example 4.

Further aspects and features of the embodiments described herein will appear from the following description taken together with the accompanying drawings.

### DETAILED DESCRIPTION OF THE EMBODIMENTS

Various compositions, systems and methods will be described below to provide an example of at least one embodiment of the claimed subject matter. No embodiment described below limits any claimed subject matter and any claimed subject matter may cover compositions, systems, devices and methods that differ from those described below. The claimed subject matter is not limited to compositions, systems, devices and methods having all of the features of any one composition, system. device or method described below or to features common to multiple or all of the compositions, systems, devices and methods described below. It is possible that any composition, system, device or method described below is not an embodiment of any claimed subject matter. Any subject matter that is disclosed in a composition, system, device or method described below that is not claimed in this document may be the subject matter of another protective instrument, for example, a continuing patent application, and the applicants, inventors or owners do not intend to abandon, disclaim or dedicate to the public any such subject matter by its disclosure in this document.

Furthermore, it will be appreciated that for simplicity and clarity of illustration, where considered appropriate, reference numerals may be repeated among the figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures and components have not been described in detail so as not to obscure the embodiments described herein. Also, the description is not to be considered as limiting the scope of the embodiments described herein.

Hydrogel System

Gelatin is a biopolymer prepared by thermal denaturalization and hydrolysis of collagen. Hydrolysis results in the reduction of collagen protein fibrils of about 300,000 Da into smaller peptides. Depending upon the process of hydrolysis, peptides will have broad molecular weight ranges associated with physical and chemical methods of denaturation. Gelatin contains enzymatically degradable sites and cell binding domains, making it an attractive biomaterial in tissue engineering applications, for example, as a cell scaffold material in tissue repair.

Photocurable gelatin hydrogels contain chemically-modified (functionalized) gelatin polymers capable of chemical crosslinking, for example, in the presence of a photoinitiator. Examples of chemically-modified gelatin include, but are not limited to, methacrylated gelatin, acrylated gelatin and thiolated gelatin. The properties of photocurable gelatin hydrogels can be tuned by adjusting various parameters of the gelatin itself or during material processing according to methods known to those skilled in the art.

Methacrylated gelatin (GelMA) is an inexpensive, biocompatible, photocrosslinkable material that can be degraded by matrix metalloproteinases (MMP), which are produced at increased levels during wound healing. GelMA hydrogels are typically used in tissue engineering applications, e.g. as a cell scaffold, where the gel must be sufficiently porous to encapsulate whole cells and to permit nutrients and large molecules to diffuse through. GelMA is typically considered too soft and porous for use in drug delivery applications, as it is considered too diffusive to retain therapeutic molecules.

As disclosed herein, GelMA formed using a method comprising sequential physical and chemical crosslinking steps (GelMA+) was surprisingly found to have physical, mechanical and biodegradable properties suitable for drug delivery applications, in particular, for applications involving enzyme-mediated drug release. GelMA+ was able to sustain the release of a payload over a prolonged period of time. GelMA+ performed far superior to standard GelMA in this regard. The GelMA+ system was able to incorporate and release payloads of different sizes and could be tuned to adjust the physical, mechanical and biodegradable characteristics of the hydrogel. Furthermore, drug release could be further controlled with the use of enzymes capable of degrading the hydrogel.

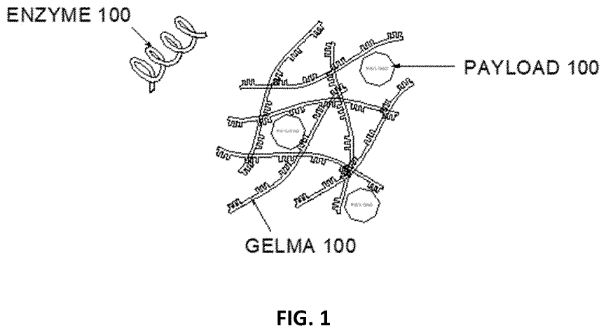

The present disclosure relates generally to a gelatin hydrogel system useful for delivering various payloads. Referring now to FIG. 1, shown therein is an example of the GelMA+ payload delivery system consisting of GelMA+ (GELMA 100), the payload (PAYLOAD 200), and an enzyme trigger (ENZYME 200). In some embodiments, there is disclosed a hydrogel system for delivering a payload, which system comprises a gelatin hydrogel formed by a method comprising sequential physical and chemical crosslinking steps; and a payload. The gelatin comprises chemically-modified gelatin polymers capable of photocrosslinking. In some embodiments, the modified gelatin polymers comprises methacrylated gelatin, acrylated gelatin, thiolated gelatin or a combination thereof. In particular embodiments, the modified gelatin comprises methacrylated gelatin. In some embodiments, the chemically-modified gelatin may be further modified to enhance desired characteristics.

The hydrogel is formed by a method involving sequential crosslinking steps, in particular, physical and chemical crosslinking steps. By “sequential,” it is meant that one step in the sequence is initiated before the other. In some embodiments, the earlier step in the sequence is completed prior to initiating the subsequent step in the sequence. In some embodiments, there may be partial overlap wherein the subsequent step is initiated prior to total completion of the earlier step in the sequence. In some embodiments, the physical crosslinking step is completed prior to initiation of the chemical crosslinking step.

As used herein, “GelMA” refers gelatin methacrylate (or methacrylated gelatin or gelatin methacryloyl) and may be used to refer to gelatin methacrylate prior to crosslinking or to gelatin methacrylate crosslinked using a conventional method of UV crosslinking without a prior physical crosslinking step. The term “GelMA+” refers to gelatin methacrylate crosslinked using a method comprising sequential physical and chemical crosslinking steps.

GelMA may be prepared by any suitable means, including those disclosed herein. For example, GelMA may be prepared by treating gelatin with methacrylic anhydride. The degree of methacrylation may vary, for example a high degree of methacrylation may be considered to be about 80% or higher, for example about 90% or higher, and a low degree of methacrylation may be considered to be about 50% or lower. However, any suitable degree of methacrylation could be used, provided the methacrylated gelatin achieves sequential physical and chemical crosslinking to form a hydrogel system suitable for payload delivery, as described herein. The methacrylation degree may be between about 30% to about 90%, such as between about 40% to about 90%, between about 50% to about 90%, between about 60% to about 80%, between about 50% to about 60%, between about 60% to about 70%, between about 70% to about 80%, between about 80% to about 90%, between about 90% to about 95%, between about 90% to about 99%, for example, about 30%, about 40%, about 50%, about 60%, about 70%, about 80%, about 90%, about 95%, or about 99%. It will be understood that the conditions for preparing GelMA can be used to tune the properties of the GelMA hydrogel system. For example, the degree of methacrylation, the conditions of the physical and chemical crosslinking steps, such as the concentration of GelMA, could be tuned. A controlled release profile may be tuned by altering, for example, the porosity, crosslinking site density, and permeability of the hydrogel system by varying the previous or other parameters.

It will be understood that gelatin could be functionalized with a group other than methacrylate to achieve a hydrogel system for delivering a payload without departing from the spirit of the present invention. It will be understood that other functional groups capable of functionalizing and crosslinking gelatin could be used.

It will be understood that other hydrogels formed from sequential physical and chemical crosslinking steps could be used to achieve a hydrogel system for delivering a payload without departing from the spirit of the present invention. For example, collagen may be used in place of gelatin to achieve a hydrogel system for delivering a payload. Sequential physical and chemical crosslinking as described herein could be done on other hydrogels having physical crosslinking, induced by incubation at a given temperature, and chemical crosslinking functionalities. For example, other proteins which undergo a sol-gel transformation upon cooling (thermal gelation or thermal crosslinking) and also contain a functionality compatible with chemical crosslinking (e.g. a methacrylated, acrylated, or thiolated protein) could also be used in the hydrogel system of the present disclosure.

The hydrogel system of the present disclosure may include one or more additional components, such as one or more other polymers. Suitable polymers for incorporation in to hydrogel systems are know to those skilled in the art. The one or more polymers could include, for example, gelatin methacrylate, carboxybetaine methacrylate (CBMA), Alginate hydrogel, poly(hydroxylethylmethacrylate) (HEMA), Collagen derivatives, Poly lactic glycolic acid (PLGA), acrylamide gels, or any other suitable gel.

Hybrid hydrogel systems are also possible, such as: hydrogels which include gene delivery vehicles or polyplex nanoparticles; hydrogels including quantum dots for imaging; hydrogels including hydroxyapatite to induce bone growth; hydrogels as a co-delivery system to carry more than one payload; or hydrogels for secondary controlled release microparticles. It will be understood that the inclusion of other common ingredients in the hydrogel systems, such as excipients, is also possible.

The hydrogel or the hydrogel system may optionally be further processed according to methods known to those skilled in the art.

In some embodiments, the hydrogel system may be dried, frozen, or lyophilized and stored prior to use. The hydrogel may be lyophilized before or after encapsulation of the payload, and subsequently reconstituted prior to further processing or end use.

Physical Crosslinking

The term “physical crosslinking” as used herein refers to physical association of functionalized gelatin under suitable conditions and for a sufficient period of time to allow self-assembly of at least a portion of the functionalized gelatin. Physical crosslinking may also be considered physical gelation. The self-assembly of functionalized gelatin molecules may be stabilized through hydrogen bonding and/or electrostatic interactions. Physical crosslinking may cause assembly of gelatin chains into a partial triple helical configuration. In some embodiments, a cool solution of GelMa is incubated for a period of time (an incubation period) sufficient to permit physical crosslinking. It will be understood that the conditions for physical crosslinking demonstrated herein were exemplary in nature, and the conditions for physical crosslinking could be altered without departing from the sprit of the present invention. Without wishing to be bound by theory, it is believed that the physical crosslinking step reduces the porosity of the resultant hydrogel system as compared to a hydrogel system prepared with chemical crosslinking only.

Physical crosslinking may be carried out on a solution containing any suitable amount of the modified gelatin. In some embodiments, the physical crosslinking step is carried out on methacrylated gelatin in solution. In some embodiments, the solution comprises about 1-50% (w/v), or about 1-35% of modified gelatin, for example between about 1%-5%, 1%-10%, 5%-30%, 10%-35%, 10%-30%, 10%-20%, 20%-30%, 25%-35% or about 1%, 5%, 10%, 15%, 20%, 25%, 30%, or 35% modified gelatin. Physical crosslinking may be carried out in an aqueous solution and/or a physiologically compatible diluent or carrier. In some embodiments, the physical crosslinking may be carried out in phosphate buffered saline (PBS). Air may optionally be removed from the solution, which may improve the physical properties of the resultant hydrogel system.

The physical crosslinking step may be carried out for any suitable amount of time. In some embodiments, the physical crosslinking step may involve an incubation period of at least 30 minutes, at least 45 minutes, or at least 1 hour. In some embodiments, physical crosslinking may involve an incubation period of between about 15 minutes—about 3 hours, between about 30 minutes to about 2 hours, between about 45 minutes to about 1.5 hours, or about 15 minutes, about 30 minutes, about 45 minute, about 1 hour, about 1.5 hours, about 2 hours, or about 3 hours. In some embodiments, physical crosslinking may be carried out until the storage modulus of the gel exceeds the loss modulus when measured using rheology. In some embodiments, physical crosslinking may be carried out until the hydrogel passes a “vial inversion test”. In particular, when, upon inversion, the GelMA solution does not flow, it has passed the vial inversion test.

Physical crosslinking may be carried out at an any suitable incubation temperature. In some embodiments, the incubation temperature is between about 1-16° C. In some embodiments, physical crosslinking may carried out at an incubation temperature between about 2-15° C., between about 2-10° C., between about 3-8° C., between about 4-6° C., between about 3-5° C. or about 2° C., 3° C., 4° C., 5° C., 6° C., 7° C., 8° C., 10° C., 12° C., 14° C. or 16° C. In some embodiments, physical crosslinking is carried out at an incubation temperature of about 4° C.

It will be understood that the various conditions for physical crosslinking may be tuned. For example, a higher concentration solution may require a shorter incubation period, or an increased temperature may require a longer incubation period.

Chemical Crosslinking

The term “chemical crosslinking” as used herein refers to any means of facilitating the formation of a covalent bond between the components of the hydrogel system, such as between modified gelatin molecules, for example, methacrylated gelatin molecules. It will be understood that the conditions for chemical crosslinking used in the exampled herein were exemplary in nature, and the conditions for chemical crosslinking could be altered without departing from the sprit of the present invention. In some embodiments, the chemical crosslinking step may involve a catalyst. Chemical crosslinking may involve thermal and/or photochemical reactions. In some embodiments, chemical crosslinking may involve glutaraldehyde or click chemistry-based crosslinking, such as thiol-ene crosslinking. In some embodiments, chemical crosslinking may involve UV-induced crosslinking of GelMA. In such embodiments, chemical crosslinking is carried out with UV irradiation, such as UV irradiation in the presence of a photoinitiator. It will be understood that any suitable photoinitiator may be used. In some embodiments, the photoinitiator may be 2-hydroxy-4′-(2-hydroxyethoxy)-2-methylpropiophenone (Irgacure 2959, or IC2959). The photoinitiator may be lithium phenyl-2,4,6-trimethylbenzoylphosphinate (LAP), 2,2′-azobis[2-methyl-n-(2-hydroxyethyl)propionamide] (VA-086), 2′,4′,5′,7′-tetrabromofluorescein disodium salt (Eosin Y), or any other suitable UV activated photoinitiator. The UV irradiation may be applied for any suitable amount of time. In some embodiments, the UV irradiation is applied for between about 10 seconds to about 30 minutes, for example between about 10 seconds to about 30 seconds, between about 30 seconds to about 90 seconds, between about 10 seconds to about 1 minute, between about 1 minute to 5 minutes, between about 1 minute to about 2 minutes, between about 2 minutes to about 5 minutes, between about 5 minutes to about 10 minutes, between about 10 minutes to about 20 minutes, or about 10 seconds, 30 seconds, 60 seconds, 90 seconds, 2 minutes, 3 minutes, 4 minutes, 5 minutes, 10 minutes, 15 minutes, 20 minutes, or 30 minutes. In embodiments comprising a photoinitator, the irradiation may be of any wavelength of light suitable to activate the photoinitiator. In some embodiments, chemical crosslinking may be carried out with UV irradiation between about 360-480 nm, such as between about 360-450 nm, between about 380-480 nm, between about 400-450 nm, between about 360-400 nm, or about 360 nm, 370 nm, 380 nm, 390 nm, 400 nm, 410 nm, 420 nm, 430 nm, 440 nm, 450 nm, 460 nm, 470 nm, or 480 nm. Chemical crosslinking may include UV irradiation at any suitable intensity, for example about 8 mW/cm² to about mW/cm², such as 32 mW/cm², 225 mW/cm², or 2700 mW/cm².

It will be understood that the particular conditions for chemical crosslinking will be tunable based on the hydrogel system, including the type of functional groups within the hydrogel, as well as whether thermal or photochemical crosslinking takes place. For UV-initiated crosslinking, for example, the amount of time required may be less with higher intensity light, or vice versa.

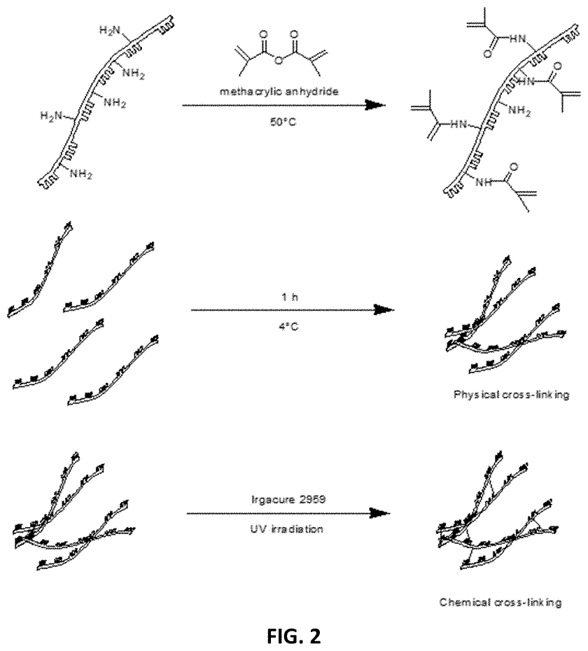

Referring now to FIG. 2, shown therein is an example process for the fabrication of GelMA+. GelMA is synthesized by the direct reaction of Type A Gelatin (10% w/v) with Methacrylic Anhydride (MA) in phosphate buffer (pH=7.4) at 50° C. This reaction introduces the methacryloyl substitution groups on the reactive amine and the hydroxyl groups of the amino acid residues. The resultant product is washed with PBS and lyophilized to obtain freeze dried GelMA. To the freeze dried GelMA, a photoinitiator (Irgacure 2959) is added and then it is incubated at 4° C. for one hour. The mixture is then physically crosslinked before being UV crosslinked, where it undergoes photoinitiated radical polymerization to form a covalently crosslinked hydrogel that is GelMA+.

Payload

The hydrogel system as described herein, which includes a crosslinked hydrogel formed by a method comprising sequential physical and chemical crosslinking steps, is useful for delivering payload. A wide variety of payloads can be loaded into the hydrogel system described herein. At least a portion of the payload may be encapsulated within a matrix formed by the hydrogel. In some embodiments, at least about 50% to about 99% of the payload is encapsulated within a matrix formed by the hydrogel, for example, at least about 50%, 60%, 70%, 75%, 80%, 85%, 90%, 95%, 97%, 98% or 99%. In some embodiments, a portion of the payload may be associated with a surface of the hydrogel and/or covalently linked to the hydrogel. For example, in some embodiments, the payload can be a pharmaceutical covalently linked to the hydrogel.

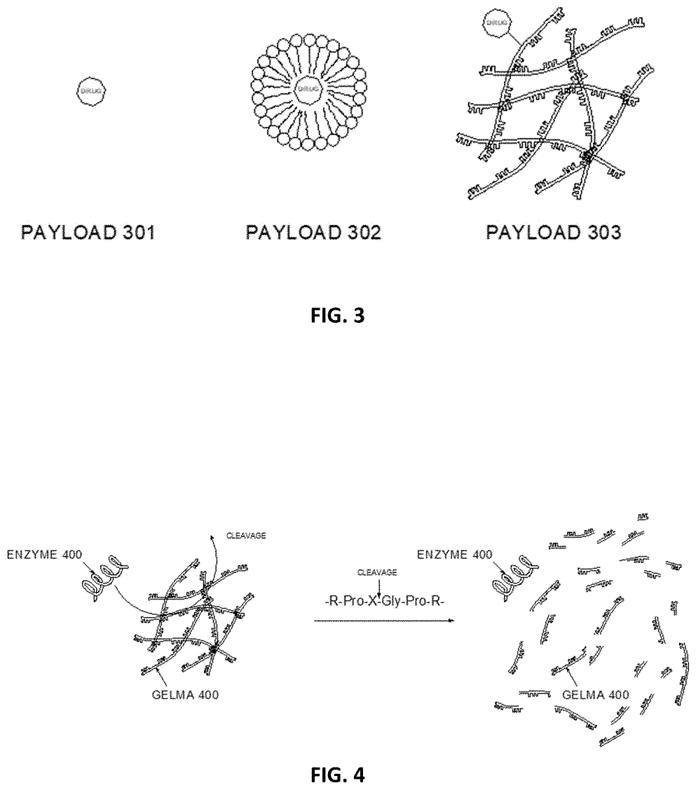

Referring now to FIG. 3, shown therein is an example of a payload (PAYLOAD 301), which may be any suitable payload as described herein. In some embodiments, the payload is a payload that has been encapsulated in another delivery vehicle such as a nanoparticle or liposome (PAYLOAD 302). In some embodiments, the payload is covalently attached to the hydrogel (PAYLOAD 303). In the case of PAYLOAD 303, the payload may have a modified active group that can be chemically crosslinked with the hydrogel mixture.

As used herein, a “payload” refers to any agent of interest capable of be delivered using the hydrogel delivery system of the present invention. In some embodiments, the payload may be a therapeutic agent (ex. drug), a preventative agent (e.g. a vaccine), a marker, or even a cell. In some embodiments, the payload may be a protein, a peptide, an antibody, a nucleic acid molecule, or a carbohydrate. In some embodiments, the payload may comprise a small molecule. In some embodiments, the payload may comprise a biologic molecule.

In some embodiments, the payload is a single agent of interest. In other embodiments, the payload comprises more than one agent of interest, for example, a combination of two or more agents of interest. In some embodiments, the payload comprises 2, 3 or 4 agents of interest. For example, the payload may comprise two or more compatible agents of interest selected from a therapeutic agent, a preventive agent, a marker, a cell, a protein, a peptide, an antibody, a nucleic acid molecule, or a carbohydrate.

In some embodiments, the payload may comprise one or more agents of interest incorporated or encapsulated in another vehicle, such as a nanoparticle, nanowire, nanotube, liposome, or micelle, for delivery of the agent of interest via the hydrogel system disclosed herein.

The payload may be selected or designed such that it is sufficiently large that all or a desired portion of the payload is capable of being retained within a matrix formed by the hydrogel until degradation of the hydrogel occurs or is initiated. In some embodiments, the payload has a molecular weight of the payload is at least about 1 kDa, at least about 2 kDa, at least about 4 kDa, at least about 10 kDa, at least about 15 kDa, at least about 30 kDa, at least about 50 kDa, at least about 70 kDa, at least about 100 kDa. In some embodiments, the payload has a molecular weight of less than about 100 kDa, less than about 70 kDa, less than about 50 kDa, less than about 30 kDa, less than about 15 kDa, less than about 10 kDa, less than about 4 kDa, less than about 2 kDa. In some embodiments, the payload may have a molecular weight between about 1 kDa and about 1000 kDa, between about 2 kDa to about 100 kDa, between about 3 kDa to about 80 kDa, between about 4 kDa to about 70 kDa, between about 1 kDa to about 50 kDa, or between about 50 kDa to about 100 kDa.

In some embodiments, the payload is a therapeutic agent. In some embodiments, therapeutic agent is useful in promoting wound healing. Suitable therapeutic agents will be known to a person of skill in the art. Some such agents are outlined, for example, in Son, Y. J. et al. “Biomaterials and controlled release strategy for epithelial wound healing” Biomaterials Science 2019, 7, 4444, the entire contents of which are incorporated herein by reference. The therapeutic agent may be a growth factor, such as Epidermal Growth Factor (EGF), Heparin-binding EGF (HB-EGF), Insulin-like Growth Factor (IGF-1), Epiregulin, Platelet-derived growth factor α and β (PDGF-α/β), Transforming growth factor α (TGF-α), Transforming growth factor β (TGF-β), Keratinocyte growth factor (KGF), Hepatocyte growth factor (HGF), or Fibroblast Growth Factor (FGF). In some embodiments, the therapeutic agent is an extracellular matrix (ECM) component, a cytokine, a growth factor or a drug. In some embodiments, the payload is an antimicrobial agent (such as an antibacterial, antifungal or antiviral). In some embodiments, the payload is hyaluronic acid, bovine lactoferrin, Epidermal Growth Factor (EGF), Heparin-binding EGF (HB-EGF), Insulin-like Growth Factor (IGF-1), Epiregulin, Platelet-derived growth factor α and β (PDGF-α/β), Transforming growth factor α (TGF-α), Transforming growth factor β (TGF-β), Keratinocyte growth factor (KGF), Hepatocyte growth factor (HGF), or Fibroblast Growth Factor (FGF).

Enzyme

The hydrogel system of the present disclosure is enzyme-degradable, meaning that degradation of the hydrogel may be facilitated or enhanced by one or more enzymes. As will be appreciated by those skilled in the art, the particular enzyme/s capable of degrading the hydrogel will depend on the components and properties of the hydrogel. Different enzymes may degrade the hydrogel differently, such as, at differing rates, or by differing mechanisms. In some embodiments, the enzyme may degrade the hydrogel entirely. Is some embodiments, the enzyme may increase the porosity of the hydrogel. In some embodiments, the enzyme is an enzyme capable of degrading a gelatin-based hydrogel. In some embodiments, the enzyme is an extracellular matrix-degrading enzyme. In some embodiments, the enzyme is a matrix metalloproteinase (MMP), such as a collagenase or gelatinase. In some embodiments, the MMP is MMP-2, MMP-8, or MMP-9. In some embodiments, the enzyme is an enzyme present at a wound site. In some embodiments, the enzyme is an enzyme that is upregulated at a wound site. In some embodiments, the enzyme is added to the hydrogel externally. The enzyme may be any suitable enzyme, or combination of enzymes, capable of degrading the hydrogel at a suitable rate and/or over a suitable amount of time. The choice of hydrogel components, or the choice of enzyme/s to facilitate degradation, may be guided by the particular application. For example, some MMPs are knows to be upregulated at sites of wound healing. For example, there is upregulation of MMPs in diseases such rheumatoid arthritis, osteoarthritis, teeth and gum infections, tumor invasion and progression, acute and chronic wounds. During pregnancy, there is also an increase in the production of MMPs. Since MMPs are know to degrade gelatin, MMPs present at wound healing sites may assist in degrading a gelatin-based hydrogel.

In some embodiments, an enzyme is applied eternally to the hydrogel system permit further tuning of hydrogel degradation and payload release profiles. The enzyme may be added to the hydrogel system by any suitable means. The enzyme, or combination of enzymes, may be selected based on, for example, desired degradation profile of the hydrogel and/or desired payload release profile. For example, MMP-8 and MMP-9 were shown to degrade GelMA hydrogel systems at differing rates, as described herein. The slower degradation of GelMA+ by MMP-9 could be particularly useful in degrading the hydrogel to achieve a delayed release of payload. In other applications, the more efficient degradation of GelMA+ by MMP-8 may be used to achieve a faster payload release. Other hydrogel-degrading enzymes could be selected to achieve a desired hydrogel degradation and/or payload release profile for a particular application, without departing from the spirit of this invention.

Matrix metalloproteinases may be present in vivo, for example, MMP-9 has been observed to be up-regulated (with an elevated level) in various types of wounds, including ocular wounds, skin wounds, chronic skin wounds. This evaluated physiological level may be an advantage to be a stimulus for a stimulus-responsive (enzyme-responsive) and sustained release system, such as the hydrogel system described herein. In particular, the experiments disclosed herein suggest the hydrogel system can be degraded by physiologically relevant concentrations of matrix metalloproteinases, such as MMP-8 and MMP-9 levels present at a wound site.

The secretion and activity of MMPs are highly regulated. In normal tissues, the expression of MMPs are at a basal level, but in wounded tissues the expression of the MMPs increase rapidly and get activated. Different cells within the skin such as keratinocytes, fibroblasts, endothelial cells and inflammatory cells like macrophages, lymphocytes and monocytes express MMPs. A range of signal like cytokines, hormone, or extracellular matrix induce the expression of MMPs. Growth factors like epidermal growth factor (EGF), hepatocyte growth factor (HGF), fibroblast growth factor (FGF), vascular endothelial growth factor (VEGF), platelet-derived growth factor, tumor necrosis factor-alpha (TNF-a), keratinocyte growth factor (KGF), transforming growth factor-beta (TGF-b), as well as interleukins and interferons activate MMPs.

In some embodiments, the enzyme may be added to the hydrogel system externally, such as after the hydrogel has been administered to a wound. In such an embodiment, it may be advantageous to use a more efficiently hydrogel-degrading enzyme, such as MMP-8. Administering an enzyme, such as MMP-8 or MMP-9, to the hydrogel encompasses any suitable way to apply the enzyme to the hydrogel. If the hydrogel system was applied to a wound site, it may be preferable to apply the enzyme directly to the hydrogel system rather than to the wound itself.

The enzyme may degrade the hydrogel quickly or slowly. The hydrogel system of the present disclosure may be optimized to release a particular payload over a particular release period. The release period may be over a period of hours, days, weeks, or months. In some embodiments, the release period may be up to about 1 day, up to about 5 days, up to about 7 days, up to about 10 days, up to about 14 days, or more. In some embodiments, the release period may be between about 12-24 hours, between about 24 to 48 hours, between about 48 to 72 hours, between about 1-2 days, between about 1-5 days, between about 1-14 days, between about 4-10 days, between about 7-10 days, between about 7-14 days, or between about 1-30 days, or about 1 day, about 2 days, about 3 days, about 4 days, about 5 days, about 6 days, about 7 days, about 10 days, about 14 days, about 21 days, or about 28 days. In some embodiments, the release period may be between about 1-12 months, such as, between about 1-3 months, about 1-6 months, about 1-9 months, about 3-6 months, about 3-9 months, about 9-12 months, about 6-12 months, or about 1, about 2, about 3, about 5, about 7, about 9, or about 12 months.

Referring now to FIG. 4, shown therein is an example mechanism for GelMA+ (GELMA 400) degradation by a trigger enzyme (ENZYME 400), such as a matrix metalloproteinase (MMP), collagenase or gelatinase. This includes MMP9, MMP8, MMP2, which are also known as 92 kDa type 4 collagenase, Type 2 collagenase, and 72 kDa Type 4 collagenase respectively. MMP9 and MMP2 are also called Gelatinase B, and Gelatinase A respectively.

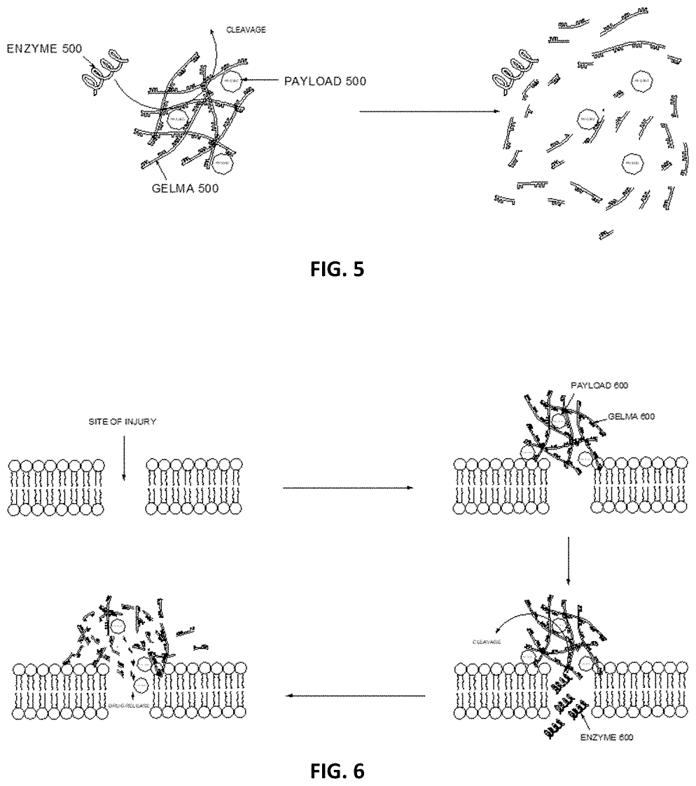

Referring now to FIG. 5, shown therein is an example mechanism for the release of the payload (PAYLOAD 500) from GelMA+ (GELMA 500) in the presence of the trigger enzyme (ENZYME 500). The GelMA+ hydrogel undergoes enzymatic degradation where the gelatin molecules are cleaved by the trigger enzymes causing release of the payload. It will be understood that any hydrogel system which is degradable by a similar or suitable mechanism could be used in the hydrogel system of the present disclosure.

Referring now to FIG. 6, shown therein is an example application of the GelMA+ payload delivery system for wound healing, comprising GelMA+(GELMA 600) and a payload (PAYLOAD 600). The GelMA+ delivery system, when introduced to the site of injury (e.g. a corneal wound), undergoes enzymatic degradation due to the presence of an enzyme (ENZYME 600) such as an MMPs with increased levels at the wound site. This enzymatic degradation leads to the release of the payload from the GelMA+ matrix to the surface of the wound, thereby enhancing wound healing.

Devices

The hydrogel system as described herein may be applied as, formed into and/or incorporated into a device suitable for delivering a payload. The device may be configured to release the payload over a release period, such as any release period as defined for the hydrogel system herein. In some embodiments, the device may be a lens, such as a contact lens, an implant, such as a corneal implant, an insert, a patch, a bandage, or a dressing. In some embodiments, the device is contacted with an enzyme to facilitate hydrogel degradation and payload release. In some embodiments, the enzyme is added to the device externally (e.g. exogenous enzyme is applied to the system as opposed to endogenous enzyme, such as an enzyme naturally present at a wound site). In some embodiments, an enzyme is added externally after the device has been applied to a subject. Exogenous enzyme may be added to the device by any suitable means.

In some embodiments, the device is a bandage, a patch, an implant, an insert or a lens. In some embodiments, the lens is a contact lens. In some embodiments, the implant is a corneal implant, although other hydrogel implants are also contemplated. In some embodiments, the insert is an ocular insert, although other hydrogel inserts are also contemplated. could be added in a liquid formulation to the bandage or contact lens for faster payload delivery.

In some embodiments, the device is a device for promoting wound healing. The hydrogel system may be incorporated into any suitable wound treatment device.

In one embodiment, the hydrogel system could be incorporated into an ocular insert to the lower eyelid pocket, and optionally, an enzyme such as an MMP could be added as an eye drop to the lower eyelid.

In one embodiment, the hydrogel system could be incorporated into a wound dressing, applied to a clean wound bed, and optionally, an enzyme such as an MMP could be added to the dressing to trigger release.

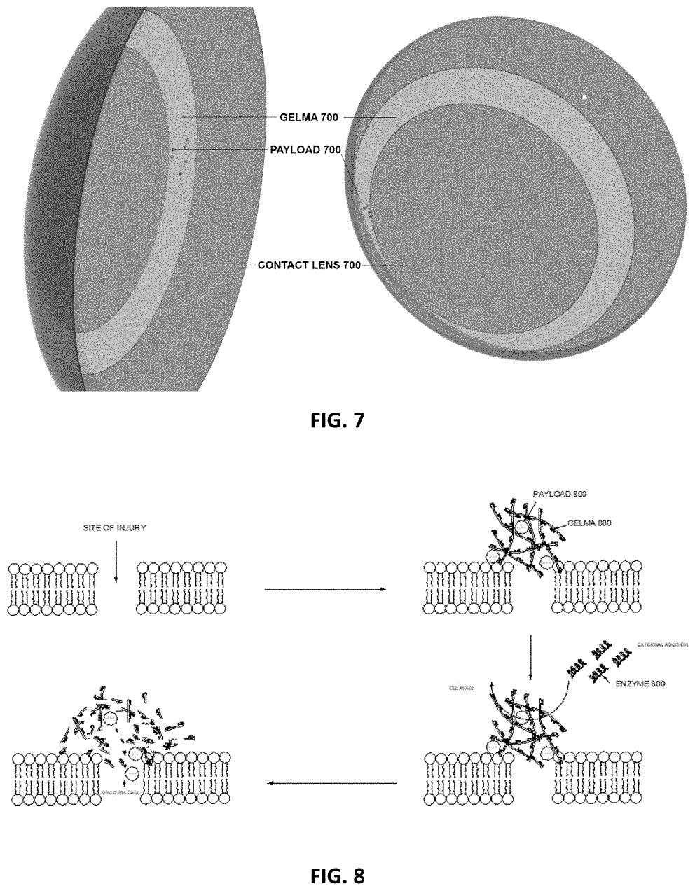

Referring now to FIG. 7, shown therein is an example application of the GelMA+ payload delivery system (GELMA 700) used in combination with another system for wound healing. The GelMA+ system is embedded as a ring on the contact lens (CONTACT LENS 700), outside the center viewing zone to allow for unobstructed vision. When the ring comes in contact with a wound, the upregulated MMPs at the site cause degradation of the GelMA+ drug delivery system, which consequently releases the payload (PAYLOAD 700).

Treatment of Wounds

The hydrogel system may be used to deliver a payload to a wound site of a subject. In some embodiments, the would may be or result from an ocular wound, a burn wound, a chemical burn wound, an acute wound, a chronic wound, a bone wound, an ulcer, a pressure ulcer, a venous ulcer, or a bedsore. In some embodiments, the subject is animal, such a mammal, such as a human. The components of the hydrogel system and any enzymes to be applied should be selected with the species and biology of the subject in mind.

Method of Making Hydrogel System and Devices

The hydrogel system may be made by any suitable method, including the methods disclosed herein and in Rizwan et al. 2017 incorporated herein)). The exemplary crosslinked gelatin methacrylate (GelMA) hydrogel described herein is obtained by a method comprising sequential physical and chemical crosslinking steps, where physical crosslinking and chemical crosslinking are as defined herein. The method of making the hydrogel system may be modified by those of skill in the art without departing from the spirit of the present invention.

The hydrogel system described herein may be incorporated into another composition or device for delivery of the payload to a subject. It will be understood in the art how to incorporate the hydrogel system into other compositions and devices.

Method of Delivering a Payload

The hydrogel system described herein is for use in delivering a payload to a subject. Many different uses and payloads are contemplated. A method of delivering a payload to a subject comprises, in general terms, administering to the subject a hydrogel system as disclosed herein. In some embodiments, administration comprises applying a composition or device comprising the hydrogel delivery system to a subject. In some embodiments, the hydrogel system is administered as or in a device as disclose herein. The hydrogel system, composition or device may be applied to the subject by any suitable means. It will be understood that, where the hydrogel system, composition or device is to be applied to a subject, the components of the final system, composition or device should be physiologically-acceptable. In some embodiments, the method is a method treating or preventing a condition or disease, e.g. a disease or condition that is treatable or preventable by administration of the payload. In some embodiments the method is a method of treating a wound, e.g. to facilitate would healing. The hydrogel system, composition or device may be used for treating any suitable wound. The payload may be loaded into the hydrogel at any suitable point in the process. In some embodiments, the payload is loaded into the hydrogel prior to physical crosslinking. In some embodiments, the payload is loaded into the hydrogel after physical crosslinking and before chemical crosslinking. In some embodiments, the payload is loaded into the hydrogel after chemical crosslinking.

In some embodiments of wound healing applications, the payload can be loaded into GelMA+ and can be used a bandage contact lens placed over the injured cornea to release payload for corneal wound healing. It can be used as an implant and/or corneal implant to be injected at the wound site to deliver the above-mentioned payload slowly on being acted upon by the matrix metalloproteinase enzymes. Similarly, payload loaded GelMA+ hydrogel can be used as a patch, bandage or wound dressing to deliver the payload at the wound surface on being degraded by the matrix metalloproteinase enzymes. The MMPs may be present at the wound site and/or may be added exogenously.

Delivery of the payload is tuneable based on various factors as will be understood by those of skill in the art. Referring to an exemplary embodiment, delivery may be tuned by factors including but not limited to the payload itself, GelMA density, methacrylation degree, crosslinking degree, or the sequential crosslinking steps. The release rates and profiles may be tuned for a particular payload by tuning various properties, such as the properties of GelMA, and/or by adding an enzyme to degrade the hydrogel system. Referring now to FIG. 8, shown therein is an example application of a GelMA+ system (GELMA 800) used for delivering a payload (PAYLOAD 800) for use in wound healing, where the addition of the enzyme trigger (ENZYME 800) is from an external source. As was demonstrated herein, MMP-8 and MMP-9 showed differing degradation behaviour with model GelMA+ systems. As exemplified herein, GelMA+ hydrogel systems were effective at drug delivery and in wound healing assays, in particular, for ocular wound healing. Such studies are shown to correlate with in vivo results. It follows that GelMA+ hydrogel system could be degraded effectively by other collagenases or gelatinases, such as MMP-2, and also that the hydrogel system could be formed from collagen or other suitable proteins. From the exemplified embodiments, it was demonstrated that GelMA+ systems as described herein could be tuned to accommodate payloads in the range of about 4 kDa to about 70 kDa. It follows that payloads of varying sizes, expanding beyond the range tested, could be delivered with the hydrogel system.

Pharmaceutical Compositions

Another aspect of disclosure is related to pharmaceutical compositions comprising a hydrogel system as defined herein, and a pharmaceutically acceptable carrier. Pharmaceutical compositions may contain one or more pharmaceutically acceptable ingredients, such as pharmaceutically-acceptable carriers, diluents and/or and excipients. Pharmaceutical compositions can be prepared in a manner well known in the pharmaceutical art. The composition may be formulated in any suitable formulation or dosage form. In some embodiments, the composition may be formulated as a gel. In some embodiments, the gel is cured. The pharmaceutical composition may further include an enzyme for degrading the hydrogel system, the enzyme being any suitable enzyme as defined herein.

### Particular Embodiments

In one embodiment, the present disclosure provides a hydrogel system for delivering a payload comprising: a hydrogel comprising a crosslinkable polymer, such as a chemically-modified biopolymer, for example, chemically-modified gelatin, the hydrogel formed by a method comprising sequential physical and chemical crosslinking steps; and a payload.

The hydrogel system of any other embodiment, wherein the chemically-modified gelatin comprises methacrylated gelatin, acrylated gelatin, thiolated gelatin or a combination thereof.

The hydrogel system of any other embodiment, wherein the chemically-modified gelatin comprises methacrylated gelatin.

The hydrogel system of any other embodiment, wherein the physical crosslinking step comprises incubating a solution comprising the crosslinkable polymer under suitable conditions and for a sufficient period of time to permit physical crosslinking of at least a portion of the crosslinkable polymer.

The hydrogel system of any other embodiment, wherein the solution comprises between about 1%-35% (w/v) of the crosslinkable polymer in a suitable diluent, e.g., about 1%-about 5%, about 1%-about 10%, about 5%-about 30%, about 10%-about 35%, about 10%-about 30%, about 10%-about 20%, about 20%-about 30%, about 25%-about 35% or about 1%, about 5%, about 10%, about 15%, about 20%, about 25%, about 30%, or about 35%.

The hydrogel system of any other embodiment, wherein the solution is an aqueous solution.

The hydrogel system of any other embodiment, wherein the diluent is phosphate buffered saline (PBS).

The hydrogel system of any other embodiment, wherein the incubation period is at least 1 hour.

The hydrogel system of any preceding claim, wherein the incubation period is between about 15 minutes to about 3 hours, between about 30 minutes to about 2 hours, between about 45 minutes to about 1.5 hours, or about 15 minutes, about 30 minutes, about 45 minute, about 1 hour, about 1.5 hours, about 2 hours, or about 3 hours.

The hydrogel system of any other embodiment, wherein the physical crosslinking step comprises incubation at a temperature between about 1-about 16° C., between about 2-about 15° C., between about 2-about 10° C., between about 3-about 8° C., between about 4-about 6° C., between about 3-about 5° C. or about 2° C., about 3° C., about 4° C., about 5° C., about 6° C., about 7° C., about 8° C., about 10° C., about 12° C., about 14° C., or about 16° C.

The hydrogel system of any other embodiment, wherein the chemical crosslinking step is a thermal process.

The hydrogel system of any other embodiment, wherein the chemical crosslinking step comprises UV irradiation.

The hydrogel system of any other embodiment, wherein the UV irradiation takes place in the presence of a photoinitiator.

The hydrogel system of any other embodiment, wherein photoinitiator is selected from the group consisting of 2-hydroxy-4′-(2-hydroxyethoxy)-2-methylpropiophenone (Irgacure 2959, or IC2959); lithium phenyl-2,4,6-trimethylbenzoylphosphinate (LAP); 2,2′-azobis[2-methyl-n-(2-hydroxyethyl)propionamide] (VA-086); or 2′,4′,5′,7′-tetrabromofluorescein disodium salt (Eosin Y).

The hydrogel system of any other embodiment, wherein the chemical crosslinking step comprises UV irradiation for between about 10 seconds to about 30 minutes, for example between about 10 seconds to about 30 seconds, between about 30 seconds to about 90 seconds, between about 10 seconds to about 1 minute, between about 1 minute to about 5 minutes, between about 1 minute to about 2 minutes, between about 2 minutes to about 5 minutes, between about 5 minutes to about 10 minutes, between about 10 minutes to about 20 minutes, or about 10 seconds, 30 seconds, 60 seconds, 90 seconds, 2 minutes, 3 minutes, 4 minutes, 5 minutes, 10 minutes, 15 minutes, 20 minutes, or 30 minutes.

The hydrogel system of any other embodiment, wherein the chemical cross-linking step comprises UV irradiation with between about 360-480 nm, such as between about 360-450 nm, between about 380-480 nm, between about 400-450 nm, between about 360-400 nm, or about 360 nm, 370 nm, 380 nm, 390 nm, 400 nm, 410 nm, 420 nm, 430 nm, 440 nm, 450 nm, 460 nm, 470 nm, or 480 nm.

The hydrogel system of any other embodiment, wherein the hydrogel comprises a polymer consisting of gelatin methacrylate.

The hydrogel system of any other embodiment, wherein gelatin methacrylate is the sole polymer forming the matrix of the hydrogel.

The hydrogel system of any other embodiment, wherein the hydrogel comprises one or more additional polymers.

The hydrogel system of any other embodiment, wherein the one or more additional polymers is selected from hydrogel polymers, carboxybetaine methacrylate (CBMA), Alginate hydrogel, poly(hydroxylethylmethacrylate) (HEMA), Collagen derivatives, Poly lactic glycolic acid (PLGA), or Acrylamide gels.

The hydrogel system of any other embodiment, wherein at least a portion of the payload is encapsulated within a matrix formed by the hydrogel.

The hydrogel system of any other embodiment, wherein at least about 50-99% of the payload is encapsulated within a matrix formed by the hydrogel, for example, at least about 50%, 60%, 70%, 75%, 80%, 85%, 90%, 95%, 97%, 98%, or 99%.

The hydrogel system of any other embodiment, wherein a portion of the payload is associated with a surface of the hydrogel.

The hydrogel system of any other embodiment, wherein the payload is a therapeutic agent, preventative agent, marker, cell, or the aforementioned members encapsulated in another delivery vehicle such as a nanoparticle or liposome.

The hydrogel system of any other embodiment, wherein the payload is a small molecule or biologic molecule.

The hydrogel system of any other embodiment, wherein the payload is sufficiently large such that all or a portion of the payload is retained within a matrix formed by the hydrogel until degradation of the hydrogel occurs.

The hydrogel system of any other embodiment, wherein the molecular weight of the payload is less than about 100 kDa, less than about 70 kDa, less than about 50 kDa, less than about 30 kDa, less than about 15 kDa, less than about 10 kDa, less than about 4 kDa, or less than about 2 kDa.

The hydrogel system of any other embodiment, wherein the molecular weight of the payload is at least about 1, 2, 4, 10, 15, 30, 50, 70, or 100 kDa.

The hydrogel system of any other embodiment, wherein the payload comprises a small molecule encapsulated in another delivery system.

The hydrogel system of any other embodiment, wherein the payload comprises a small molecule encapsulated in a nanoparticle, nanowire, nanotube, liposome, or micelle.

The hydrogel system of any other embodiment, wherein the payload comprises a protein, peptide, nucleic acid, antibody, or carbohydrate.

The hydrogel system of any other embodiment, wherein the payload comprises a drug or therapeutic.

The hydrogel system of any other embodiment, wherein the payload is selected from the group consisting of an extracellular matrix component, a cytokine or a growth factor, an antimicrobial, for example, hyaluronic acid, bovine lactoferrin, Epidermal Growth Factor (EGF), Heparin-binding EGF (HB-EGF), Insulin-like Growth Factor (IGF-1), Epiregulin, Platelet-derived growth factor α and β (PDGF-α/β), Transforming growth factor α (TGF-α), Transforming growth factor β (TGF-β), Keratinocyte growth factor (KGF), Hepatocyte growth factor (HGF), or Fibroblast Growth Factor (FGF).

The hydrogel system of any other embodiment, wherein the hydrogel is degradable by an enzyme.

The hydrogel system of any other embodiment, wherein the enzyme is an enzyme present at a wound site.

The hydrogel system of any other embodiment, wherein the enzyme is an enzyme that is upregulated at a wound site.

The hydrogel system of any other embodiment, wherein the enzyme is an enzyme added to the hydrogel externally.

The hydrogel system of any other embodiment, wherein the enzyme is added after the hydrogel has been administered to a wound.

The hydrogel system of any other embodiment, wherein the enzyme is an extracellular matrix-degrading enzyme.

The hydrogel system of any other embodiment, wherein the enzyme is a matrix metalloproteinase.

The hydrogel system any other embodiment, wherein the enzyme is selected from the group consisting of MMP-2, MMP-8, and MMP-9.

The hydrogel system of any other embodiment, wherein the enzyme is MMP-8.

The hydrogel system of any other embodiment, wherein the enzyme is MMP-9.

The hydrogel system of any other embodiment, wherein the system is tuneable based on GelMA density, methacrylation degree, crosslinking degree, or sequential crosslinking steps, for compatibility with payloads of different sizes and/or release rates.

The hydrogel system of any other embodiment, wherein the hydrogel is dried and stored prior to use.

The hydrogel system of any other embodiment, wherein the hydrogel is frozen and stored prior to use.

The hydrogel system of any other embodiment, wherein the hydrogel is lyophilized and subsequently reconstituted prior to use.

The hydrogel system of any other embodiment, wherein the hydrogel is lyophilized after encapsulation of the payload.

The hydrogel system of any other embodiment, wherein the hydrogel is lyophilized prior to encapsulation of the payload.

The hydrogel system of any other embodiment, wherein delivering comprises sustained release of the payload.

The hydrogel system of any other embodiment, wherein the sustained release period is over hours, days, weeks, or months.

The hydrogel system of any other embodiment, wherein the sustained release period is between about 12-24 hours, between about 1-2 days, between about 1-5 days, between about 1-14 days, between about 4-10 days, between about 7-10 days, between about 7-14 days, or between about 1-30 days, or about 1 day, about 2 days, about 3 days, about 4 days, about 5 days, about 6 days, about 7 days, about 10 days, about 14 days, about 21 days, or about 28 days.

The hydrogel system of any other embodiment, wherein the sustained release period is between about 1-12 months, such as, between about 1-3 months, about 1-6 months, about 1-9 months, about 3-6 months, about 3-9 months, about 9-12 months, about 6-12 months, or about 1, about 2, about 3, about 5, about 7, about 9, or about 12 months.

In one embodiment, the present disclosure provides an enzyme-degradable hydrogel system for delivering a payload to a wound site, the system comprising: a hydrogel comprising a crosslinkable polymer, such as a chemically-modified biopolymer, for example, chemically-modified gelatin, the hydrogel formed by a method comprising sequential physical and chemical crosslinking steps; and a payload.

In one embodiment, the present disclosure provides a device for delivering a payload, the device comprising: the hydrogel system according to any other embodiment.

The device according to any other embodiment, wherein the device is a lens, such as a contact lens, an implant, such as a corneal implant, an insert, such as an ocular insert, a patch, a bandage, or a wound dressing.

The device according to any other embodiment, wherein the device is configured to release the payload over a release period.

The device of any other embodiment, wherein the release period is about 1 to about 5 days, for example, between about 12-24 hours, between about 1-2 days, between about 1-5 days, between about 1-14 days, between about 4-10 days, between about 7-10 days, between about 7-14 days, or between about 1-30 days, or about 1 day, about 2 days, about 3 days, about 4 days, about 5 days, about 6 days, about 7 days, about 10 days, about 14 days, about 21 days, or about 28 days.

In one embodiment, the present disclosure provides a method of making a hydrogel system for delivering a payload, the method comprising: providing a crosslinkable polymer, such as a chemically-modified biopolymer, for example, chemically-modified gelatin; physical crosslinking of the crosslinkable polymer; chemical crosslinking of the crosslinkable polymer; and introducing a payload into the hydrogel so-formed.

The method of any other embodiment, wherein the payload is introduced into the hydrogel prior to physical crosslinking.

The method of any other embodiment, wherein the payload is introduced into the hydrogel prior to chemical crosslinking.

The method of any other embodiment, wherein the payload is introduced into the hydrogel after physical and chemical crosslinking.

The method of any other embodiment, wherein the chemically-modified gelatin comprises methacrylated gelatin, acrylated gelatin, thiolated gelatin or a combination thereof.

The method of any other embodiment, wherein the chemically-modified gelatin comprises methacrylated gelatin.

The method of any other embodiment, wherein physical crosslinking comprises incubating a solution comprising the crosslinkable polymer under suitable conditions and for a sufficient period of time to permit physical crosslinking of at least a portion of the crosslinkable polymer.

The method of any other embodiment, wherein the solution comprises between about 1%-35% (w/v) of the crosslinkable polymer in a suitable diluent, e.g., about 1%-about 5%, about 1%-about 10%, about 5%-about 30%, about 10%-about 35%, about 10%-about 30%, about 10%-about 20%, about 20%-about 30%, about 25%-about 35% or about 1%, about 5%, about 10%, about 15%, about 20%, about 25%, about 30%, or about 35%.

The method of any other embodiment, wherein the solution is an aqueous solution.

The method of any other embodiment, wherein the diluent is phosphate buffered saline (PBS).

The method of any other embodiment, wherein the incubation period is at least 1 hour.

The method of any other embodiment, wherein the incubation period is between about 15 minutes to about 3 hours, between about 30 minutes to about 2 hours, between about 45 minutes to about 1.5 hours, or about 15 minutes, about 30 minutes, about 45 minute, about 1 hour, about 1.5 hours, about 2 hours, or about 3 hours.

The method of any other embodiment, wherein the physical crosslinking step comprises incubation at a temperature between about 1-about 16° C., between about 2-about 15° C., between about 2-about 10° C., between about 3-about 8° C., between about 4-about 6° C., between about 3-about 5° C. or about 2° C., about 3° C., about 4° C., about 5° C., about 6° C., about 7° C., about 8° C., about 10° C., about 12° C., about 14° C., or about 16° C.

The method of any other embodiment, wherein the chemical crosslinking step is a thermal process.

The method of any other embodiment, wherein the chemical crosslinking step comprises UV irradiation.

The method of any other embodiment, wherein the UV irradiation takes place in the presence of a photoinitiator.

The method of any other embodiment, wherein photoinitiator is selected from the group consisting of 2-hydroxy-4′-(2-hydroxyethoxy)-2-methylpropiophenone (Irgacure 2959, or IC2959); lithium phenyl-2,4,6-trimethylbenzoylphosphinate (LAP); 2,2′-azobis[2-methyl-n-(2-hydroxyethyl)propionamide] (VA-086); or 2′,4′,5′,7′-tetrabromofluorescein disodium salt (Eosin Y).

The method of any other embodiment, wherein the chemical crosslinking step comprises UV irradiation for between about 10 seconds to about 30 minutes, for example between about 10 seconds to about 30 seconds, between about 30 seconds to about 90 seconds, between about 10 seconds to about 1 minute, between about 1 minute to about 5 minutes, between about 1 minute to about 2 minutes, between about 2 minutes to about 5 minutes, between about 5 minutes to about 10 minutes, between about 10 minutes to about 20 minutes, or about 10 seconds, 30 seconds, 60 seconds, 90 seconds, 2 minutes, 3 minutes, 4 minutes, 5 minutes, 10 minutes, 15 minutes, 20 minutes, or 30 minutes.

The method of any other embodiment, wherein the chemical cross-linking step comprises UV irradiation with between about 360-480 nm, such as between about 360-450 nm, between about 380-480 nm, between about 400-450 nm, between about 360-400 nm, or about 360 nm, 370 nm, 380 nm, 390 nm, 400 nm, 410 nm, 420 nm, 430 nm, 440 nm, 450 nm, 460 nm, 470 nm, or 480 nm.

The method of any other embodiment, wherein the chemically-modified gelatin consists of gelatin methacrylate.

The method of any other embodiment, wherein gelatin methacrylate is the sole polymer forming the matrix of the hydrogel.

The method of any other embodiment, wherein the hydrogel comprises one or more additional polymers.

The method of any other embodiment, wherein the one or more additional polymers is selected from hydrogel polymers, carboxybetaine methacrylate (CBMA), Alginate hydrogel, poly(hydroxylethylmethacrylate) (HEMA), Collagen derivatives, Poly lactic glycolic acid (PLGA), or Acrylamide gels.

The method of any other embodiment, wherein at least a portion of the payload is encapsulated within a matrix formed by the hydrogel.

The method of any other embodiment, wherein at least about 50-99% of the payload is encapsulated within a matrix formed by the hydrogel, for example, at least about 50%, 60%, 70%, 75%, 80%, 85%, 90%, 95%, 97%, 98%, or 99%.

The method of any other embodiment, wherein at least a portion of the payload is associated with a surface of the hydrogel.

The method of any other embodiment, wherein the payload is a drug, marker, cell, or the aforementioned members encapsulated in another delivery vehicle such as a nanoparticle or liposome.

The method of any other embodiment, wherein the payload is a small molecule or biologic molecule.

The method of any other embodiment, wherein the payload is sufficiently large such that all or a portion of the payload is retained within a matrix formed by the hydrogel until degradation of the hydrogel occurs.

The method of any other embodiment, wherein the molecular weight of the payload is less than about 100 kDa, less than about 70 kDa, less than about 50 kDa, less than about 30 kDa, less than about 15 kDa, less than about 10 kDa, less than about 4 kDa, or less than about 2 kDa.

The method of any other embodiment, wherein the molecular weight of the payload is at least about 1 kDa.

The method of any other embodiment, wherein the payload comprises a small molecule encapsulated in another delivery system.

The method of any other embodiment, wherein the payload comprises a small molecule encapsulated in a nanoparticle, nanowire, nanotube, liposome, or micelle.

The method of any other embodiment, wherein the payload comprises a protein, peptide, antibody, or carbohydrate.

The method of any other embodiment, wherein the payload comprises a drug or therapeutic.

The method of any other embodiment, wherein the payload is selected from the group consisting of hyaluronic acid, bovine lactoferrin, Epidermal Growth Factor (EGF), Heparin-binding EGF (HB-EGF), Insulin-like Growth Factor (IGF-1), Epiregulin, Platelet-derived growth factor α and β (PDGF-α/β), Transforming growth factor α (TGF-α), Transforming growth factor β (TGF-β), Keratinocyte growth factor (KGF), Hepatocyte growth factor (HGF), or Fibroblast Growth Factor (FGF).

The method of any other embodiment, wherein the hydrogel is degradable by an enzyme.

The method of any other embodiment wherein the enzyme is an enzyme present at a wound site.

The method of any other embodiment wherein the enzyme is an enzyme that is upregulated at a wound site.

The method of any other embodiment, wherein the enzyme is an enzyme added to the hydrogel externally.

The method of any other embodiment, wherein the enzyme is added after the hydrogel has been administered to a wound.

The method of any other embodiment, wherein the enzyme is an extracellular matrix-degrading enzyme.

The method of any other embodiment, wherein the enzyme is a matrix metalloproteinase.

The method of any other embodiment, wherein the enzyme is selected from the group consisting of MMP-2, MMP-8, and MMP-9.

The method of any other embodiment, wherein the enzyme is MMP-8.

The method of any other embodiment, wherein the enzyme is MMP-9.

The method of any other embodiment, wherein the system is tuneable based on one or more of GelMA density, methacrylation degree, crosslinking degree, or sequential crosslinking steps, for compatibility with payloads of different sizes and/or release rates.

The method of any other embodiment, wherein the method further comprises drying the hydrogel.

The method of any other embodiment, wherein the method further comprises freezing the hydrogel.

The method of any other embodiment, wherein the method further comprises lyophilizing the hydrogel.

The method of any other embodiment, wherein the hydrogel is lyophilized after encapsulation of the payload.

The method of any other embodiment, wherein the hydrogel is lyophilized prior to encapsulation of the payload.

The method of any other embodiment, wherein physical crosslinking comprises incubating the crosslinkable polymer between glass slides.

The method of any other embodiment, wherein air is removed from the crosslinkable polymer prior to physical and chemical crosslinking.

In one embodiment, the present disclosure provides a method of making a device for delivering a payload, the method comprising, incorporating the hydrogel system according to any other embodiment into the device.

The method according to any other embodiment, wherein the device is a lens, a contact lens, an implant, a corneal implant, a patch, a bandage, or a wound dressing.

A hydrogel formed by the method of any other embodiment.

A method of delivering a payload comprising administering the hydrogel system of any other embodiment, the device of any other embodiment, to a patient.

A method of treating a wound comprising administering the hydrogel system of any other embodiment, or the device of any other embodiment, to a wound site.

The method of any other embodiment for treating an ocular wound, a burn wound, a chemical burn wound, an acute wound, a chronic wound, a bone wound, an ulcer, a pressure ulcer, a venous ulcer, or a bedsore.

The method of any other embodiment, further comprising administering an enzyme to the hydrogel.

The method of any other embodiment, for treating a patient, such as an animal or a human.

The method of any other embodiment for treating an ocular wound, comprising applying an ocular insert comprising the hydrogel system to the lower eyelid pocket.

The method of any other embodiment, wherein an enzyme solution is added in an eye drop to the lower eyelid.

The method of any other embodiment for treating a skin wound, comprising applying a wound dressing comprising the hydrogel system to the wound.

The method of any other embodiment, wherein an enzyme solution is added to the wound dressing.

In one embodiment, the present disclosure provides use of the hydrogel system of any other embodiment, or the device of any other embodiment, for delivering a payload.

Use of the hydrogel system of any other embodiment, or the device of any other embodiment, for treating a wound.

The use of any other embodiment for treating an ocular wound, a burn wound, a chemical burn wound, an acute wound, a chronic wound, a bone wound, an ulcer, a pressure ulcer, a venous ulcer, or a bedsore.

The use of any other embodiment, further comprising administering an enzyme to the hydrogel.

The use of any other embodiment, for treating a patient, such as an animal or a human.

The use of any other embodiment, for treating an ocular wound, comprising applying an ocular insert comprising the hydrogel system to the lower eyelid pocket.

The use of any other embodiment wherein an enzyme solution is added in an eye drop to the lower eyelid.

The use of any other embodiment for treating a skin wound, comprising applying a wound dressing comprising the hydrogel system to the wound.

The use of any other embodiment, wherein an enzyme solution is added to the wound dressing.

In one embodiment, the present disclosure provides a pharmaceutical composition comprising the hydrogel system of any other embodiment and a pharmaceutically acceptable excipient.

The pharmaceutical composition of any other embodiment, wherein the composition is formulated as a gel or ointment.

The pharmaceutical composition of any other embodiment, wherein the composition is formulated as a patch, implant, or bandage.

The pharmaceutical composition of any other embodiment, further comprising an enzyme for degrading the hydrogel system.

The pharmaceutical composition of any other embodiment, wherein the enzyme is an extracellular matrix-degrading enzyme.

The pharmaceutical composition of any other embodiment, wherein the enzyme is a matrix metalloproteinase.

The pharmaceutical composition of any other embodiment, wherein the enzyme is selected from the group consisting of MMP-2, MMP-8, and MMP-9.

The pharmaceutical composition of any other embodiment, wherein the enzyme is MMP-8.

The pharmaceutical composition of any other embodiment, wherein the enzyme is MMP-9.

In one embodiment, the present disclosure provides use of the pharmaceutical composition of any other embodiment, for treating a wound.

It will be understood that the hydrogel system for delivering a payload, including a hydrogel formed by a method comprising sequential physical and chemical crosslinking steps may be prepared as in the examples disclosed herein, or it may be prepared by any other suitable means. The system may be tunable based on various parameters of the materials and processes used, for example, GelMA density, methacrylation degree, crosslinking degree, or sequential crosslinking steps, for compatibility with payloads of different sizes and/or release rates. The system may be tunable based on the enzyme used to degrade the hydrogel, its concentration, and its source (i.e. endogenous or exogenous). These modifications may be carried out to form a hydrogel system for delivering a payload, without departing from the spirit of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

As used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

In the claims, as well as in the specification above, all transitional phrases such as “comprising,” “including,” “carrying,” “having,” “containing,” “involving,” “holding,” “formed from”, “composed of,” and the like are to be understood to be open-ended, i.e., to mean including but not limited to.

Only the transitional phrases “consisting of” and “consisting essentially of” shall be closed or semi-closed transitional phrases, respectively.

The following examples are intended to illustrate certain exemplary embodiments of the present disclosure. However, the scope of the present disclosure is not limited to the following examples.

### EXAMPLES

### Example 1 Release of FITC Dextran from GELMA+ in Response to MMP9

3 different formulations of GELMA+ gels were synthesized with varying crosslinking densities—10%, 20%, and 30%. A fluorescently tagged marker, FITC-dextran, with a molecular size of 70 KDa was used as a model compound and incorporated into the gels. The gels were circular discs with a diameter of 6 mm and an approximate thickness 0.5 mm.

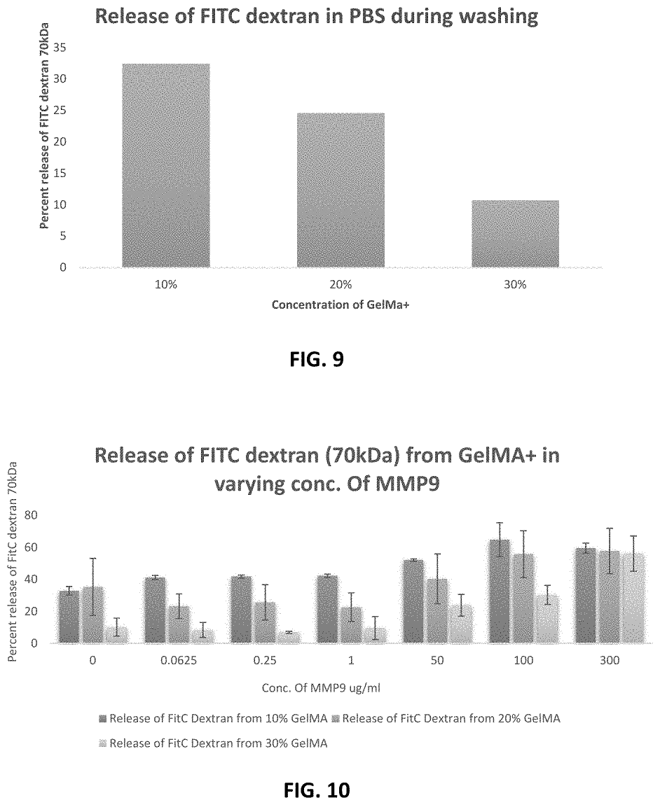

After the polymerization process, the gels were washed in PBS for 24 hours to remove any loosely bound FITC-dextran. As expected, as crosslinking density increases, the amount of loosely bound FITC-dextran decreases (FIG. 9).

When the gels were subjected to increasing concentration of MMP9 (Gibco, Thermo Fisher Scientific, MA, US) in 1 mL of PBS, from 0-300 μg/mL, the overall trend of increased release of FITC-dextran was seen (FIG. 10). This is most apparent for the 30% GELMA+ formulation.

Mechanism of Action

Due to its molecular size, 70 kDa FITC-dextran is entrapped within the GELMA+ matrix. This effect is enhanced with increasing crosslinking density of the gel. As MMP9 degrades GELMA+, FITC-dextran is released. The release increases with increasing concentration of MMP9.

### Example 2—Evaluating the Release of FITC-Dextran and Bovine Lactoferrin from a Novel Gelatin Methacrylate Hydrogel

Corneal injury, arising from surgery, disease or trauma, can cause tremendous pain that may significantly affect quality of life for the person suffering the injury. Often, it is the effect of the eyelids blinking over the corneal wound that inhibits the healing process, resulting in further complications that may result in severe vision loss. In the past, patching and ocular lubricants were the standard treatment. However, wearing an eyepatch severely limits the patient's vision, which affects their productivity. Additionally, the clinician is unable to gain any feedback on the healing process until the patch is removed.

More recently, the use of soft bandage contact lenses (BCL) overcomes both of the aforementioned problems. It effectively protects the patients from their own eyelids, while allowing the patient to continue to use their normal vision. Additionally, it also allows a clinician to view the healing of the eye. Unfortunately, current bandage contact lenses do not outperform ocular lubricants in terms of efficacy or speed of recovery, as they lack the ocular surface factors or therapeutics that are essential to aid ocular surface repair. It would, therefore, be immensely beneficial if BCLs could also deliver clinically relevant topical therapeutic drugs to the surface of the eye.

Soft contact lenses were proposed as a potential drug delivery device in the early 1960s. They are fabricated from hydrogel materials, which are three-dimensional, hydrophilic, polymeric networks capable of absorbing large volumes of fluid, as much as 20 times their molecular weight. This unique inherent property also allows these polymers to absorb and release soluble compounds such as drugs. Thus, in theory, a BCL could deliver relevant ophthalmic drugs and lubricants to facilitate corneal wound healing. However, past studies have shown that commercial contact lens materials typically are unable to maintain sustained drug release, and the vast majority of the drugs are released within the first few hours, which may not be desirable (for example see: Phan C M, Subbaraman L N, Jones L. In Vitro Uptake and Release of Natamycin from Conventional and Silicone Hydrogel Contact Lens Materials. Eye & contact lens 2013; 39:162-8; Phan C M, Subbaraman L N, Jones L. In Vitro Drug Release of Natamycin from Beta-Cyclodextrin and 2-Hydroxypropyl-Beta-Cyclodextrin-Functionalized Contact Lens Materials. Journal of biomaterials science Polymer edition 2014; 25:1907-19; Bajgrowicz M, Phan C M, Subbaraman L N, Jones L. Release of Ciprofloxacin and Moxifloxacin from Daily Disposable Contact Lenses from an in Vitro Eye Model. Investigative ophthalmology & visual science 2015; 56:2234-42; Hui A, Bajgrowicz-Cieslak M, Phan C M, Jones L. In Vitro Release of Two Anti-Muscarinic Drugs from Soft Contact Lenses. Clinical ophthalmology 2017; 11:1657-65; and/or Maulvi F A, Soni T G, Shah D O. A Review on Therapeutic Contact Lenses for Ocular Drug Delivery. Drug delivery 2016; 23:3017-26, the entire contents of which are incorporated herein by reference). Further modifications to current materials are needed to improve the release kinetics of currently available BCLs.

Among the various different types of hydrogels available, gelatin is one of the most common polymers used in biomedical applications. It is derived from hydrolysis of collagen, a naturally occurring polymer in the human cornea. Gelatin is highly biocompatible and contains a high amount of bioactive sequences, such as arginine-glycine-aspartic acid, which facilitate cell attachment. For these reasons, gelatin-based hydrogels have found wide uses in drug delivery and tissue engineering applications. However, due to its low melting point, unmodified gelatin suffers from thermal degradation (for example, see: Yue K, Trujillo-de Santiago G, Alvarez M M, et al. Synthesis, Properties, and Biomedical Applications of Gelatin Methacryloyl (Gelma) Hydrogels. Biomaterials 2015; 73:254-71, the entire contents of which are incorporated herein by reference) The weak mechanical properties of gelatin-based hydrogels can be overcome by chemical modifications, or integrating it with other monomers or polymers. Gelatin methacrylate (GelMA), a derivative of gelatin, is produced by substitution of the free amine groups of gelatin with methacrylate anhydride. This polymer can be photo-crosslinked with a photoinitiator and exposure to ultraviolet (UV) radiation to produce a permanent gel.

Interestingly, GelMA can be degraded by matrix metalloproteinases (MMP) enzymes. These enzymes, mostly MMP-2 (72 kDa type IV collagenase) and MMP-9 (92 kDa type IV collagenase) are highly upregulated in a corneal wound. Therefore, GelMA could also be used to entrap a particular drug, or drug-nanoparticles, and only release these compounds when the gel is degraded by the MMPs, which are present at a wound site. The present inventors have developed a unique transparent GelMA material, GelMA+, that was shown to have better properties for corneal tissue engineering than conventional GelMA (for example, see Rizwan M, Peh G S, Ang H-P, et al. Sequentially-Crosslinked Bioactive Hydrogels as Nano-Patterned Substrates with Customizable Stiffness and Degradation for Corneal Tissue Engineering Applications. Biomaterials 2017; 120:139-54, the entire contents of which are incorporated herein by reference). The purpose of this study is to evaluate the release of a model compound, fluorescein isothiocyanate (FitC)-Dextran, and a potential corneal wound healing therapeutic, bovine lactoferrin, from different formulation of GelMA+.

Materials and Methods

Gelatin Methacrylate Hydrogel Synthesis

Gelatin Type A, fluorescein isothiocyanate (FitC)-Dextran (70 kDa), and bovine lactoferrin (80 kDa) were obtained from Sigma Aldrich (St. Louis, Mo., USA). MMP-9 (92 kDa Type IV Collagenase) was obtained from Gibco Thermo Fisher Scientific (Grand Island, N.Y., USA). The bovine lactoferrin ELISA kit was obtained from Bethyl laboratories Inc. (Montgomery, Tex., USA)

Gelatin Methacrylate Synthesis

The method for the synthesis of GelMA+ has been previously described (for example, see Yue K, Trujillo-de Santiago G, Alvarez M M, et al. Synthesis, Properties, and Biomedical Applications of Gelatin Methacryloyl (Gelma) Hydrogels. Biomaterials 2015; 73:254-71; and/or Rizwan M, Peh G S, Ang H-P, et al. Sequentially-Crosslinked Bioactive Hydrogels as Nano-Patterned Substrates with Customizable Stiffness and Degradation for Corneal Tissue Engineering Applications. Biomaterials 2017; 120:139-54, the entire contents of which are incorporated herein by reference). In brief, 5 g of gelatin (type A) was dissolved in 50 mL of phosphate buffered saline (PBS) (10% w/v) with continuous magnetic stirring at 50-60° C. to fasten the formation of gelatin solution. 10 mL of methacrylic anhydride (20% v/v) was then added dropwise at 50-60° C. with continuous magnetic stirring. The reaction was continued for 1 hr. The resulting mixture was diluted with deionized (DI) water and dialyzed in DI water for 5 days at 40° C. using a 12-14 kDa cut-off dialysis tubes. The GelMA solution was then frozen at −80° C. and lyophilized.

Drug-Hydrogel Preparation

Lyophilized GelMA was mixed together in a PBS solution containing 0.5% w/v Irgacure 2959 to obtain a mixture with 10%, 20%, and 30% (w/v) of GelMA. The mixture was incubated at 60° C. for 48 hours. Either 60 uL of 50 mg/mL (FITC)-Dextran or 60 uL of 50 μg/mL of bovine lactoferrin was then added to the mixture and centrifuged for 5 mins at 500 rpm. The mixtures were further incubated for 30 minutes at 60° C. before carefully pipetted into a thin mould consisting of two glass microslides (thickness˜0.51 mm). The samples were then incubated at 4° C. for 1 h, before being polymerized in Dymax Ultraviolet (UV) Curing Chamber (Torrington, Conn.) for 5 minutes to create GelMA+ gels containing drugs. A circular punch was used to create discs (diameter˜6 mm) from the resulting gels.

Release Study

The samples were washed in 1 mL of PBS for 24 hours to remove any loosely bound drug. For (FITC)-Dextran release, the samples were placed in varying concentrations of MMP-9: 0 μg/mL, 0.25 μg/mL, 1 μg/mL, 10 μg/mL, 50 μg/mL, 100 μg/mL, and 300 μg/mL at room temperature (23° C.-25° C.). At t=0, 0.5, 1, 2, 4, 6, 8, 12, and 24 h, 100 μL of the release media was withdrawn and the fluorescence was read using a spectrophotometer (Molecular Devices, Sunnyvale, Calif.) at an excitation and emission wavelength of 490 nm and 520 nm. For bovine lactoferrin release, the samples were placed in varying concentrations of MMP-9: 0 μg/mL, 100 μg/mL, and 300 μg/mL. At t=0.5, 12, and 24 h, the samples were analyzed using the bovine lactoferrin ELISA kit.

Statistical Analysis

Statistical analysis and graphs were plotted using GraphPad Prism 6 software (GraphPad, La Jolla, Calif.). A repeated measures analysis of variance (ANOVA) was performed to determine the differences between gel types, time, and concentration of MMP-9. A one-way ANOVA was used to determine the differences between different concentrations of MMP-9 at 24 hours. All data was reported as mean±SD for n=3, unless otherwise stated.

Results

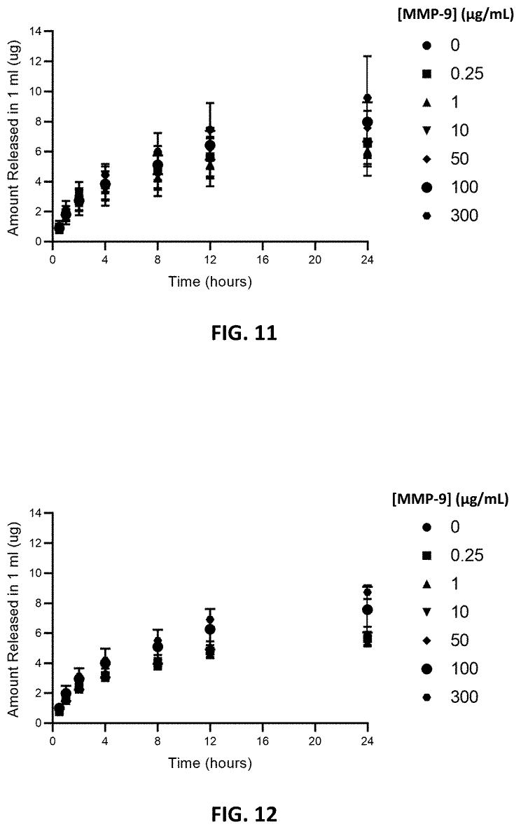

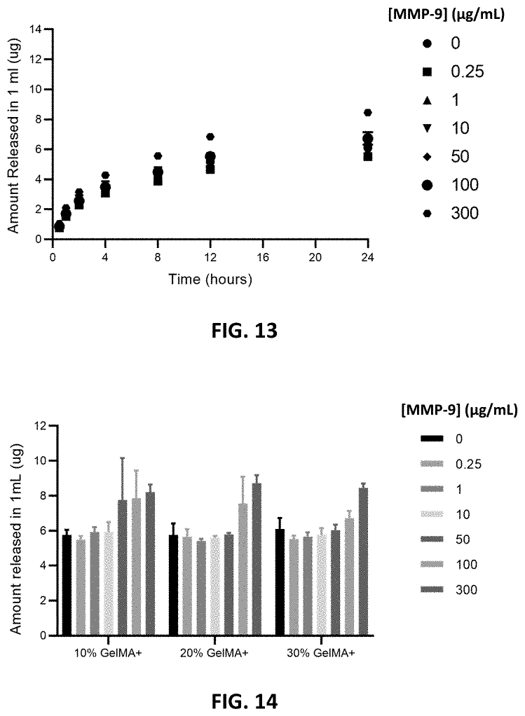

FIGS. 11, 12, and 13 show the release kinetics FITC-Dextran from 10%, 20% and 30% GelMA+ formulations respectively in varying concentrations of MMP-9 over 24 hours. The release of the FITC-Dextran was sustained over the entire 24-hour period for all the three different formulations of GelMA+ gels (p<0.05). There were no significant differences in the amount of FITC-Dextran released between the three formulations. It was noted that 10% and 20% (w/v) GelMA+ were more brittle and fragile to handle as compared to the 30% (w/v) GelMA+ gels, demonstrating that the mechanical properties of the resulting hydrogel can be modified by adjusting the concentration of GelMA+ in solution.

The cumulative amount of FITC-Dextran released after 24 hours is shown in FIG. 14. The release of FITC-Dextran increased significantly in the presence of 300 μg/mL of MMP-9 for all gel types (p<0.001).

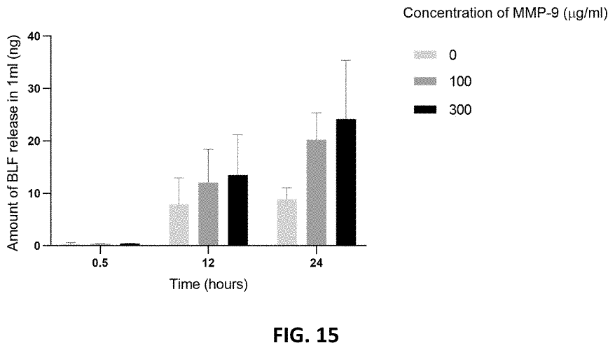

The release of bovine lactoferrin over 24 hours for 30% w/v GelMA+ is shown in FIG. 15. The release of the FITC-Dextran was sustained over the entire 24-hour period for both MMP-9 concentrations (p<0.05). For the control group, the release of bovine lactoferrin plateaued after 12 hours (p>0.05). There were also no statistically significant differences in the release at 0.5 and 12 hours between the different concentrations of MMP-9 and the control group (p>0.05). At 24 hours, there was a higher release of bovine lactoferrin due to MMP-9, but there were no significant differences between 100 and 300 μg/mL of MMP-9.

Discussion

The GelMA+ gels in the current study were able to release FITC-Dextran over the entire 24-hour testing period. Unlike other drug release studies, the release kinetics of FitC-Dextran from the gels did not show a burst release within the first hour (for example see: Phan C M, Subbaraman L N, Jones L. In Vitro Uptake and Release of Natamycin from Conventional and Silicone Hydrogel Contact Lens Materials. Eye & contact lens 2013; 39:162-8; Phan C M, Subbaraman L N, Jones L. In Vitro Drug Release of Natamycin from Beta-Cyclodextrin and 2-Hydroxypropyl-Beta-Cyclodextrin-Functionalized Contact Lens Materials. Journal of biomaterials science Polymer edition 2014; 25:1907-19; Bajgrowicz M, Phan C M, Subbaraman L N, Jones L. Release of Ciprofloxacin and Moxifloxacin from Daily Disposable Contact Lenses from an in Vitro Eye Model. Investigative ophthalmology & visual science 2015; 56:2234-42; and/or Phan C M, Subbaraman L, Liu S, et al. In Vitro Uptake and Release of Natamycin Dex-B-Pla Nanoparticles from Model Contact Lens Materials. Journal of biomaterials science Polymer edition 2014; 25:18-31, the entire contents of which are incorporated herein by reference). All gels exhibited a slow release profile for the entire 24-hour period. The inventors hypothesize that the initial wash step removed all the loosely bound FITC-Dextran on the surface or sub-surface of the gels that could have contributed to a burst release. The slower release is likely due to passive diffusion of the FITC-Dextran from within the polymer matrix.

The results for FITC-Dextran release with varying concentrations of MMP-9 suggest that there was no significant difference in the release of FITC-Dextran for low concentrations of MMP-9. This indicates that that the release of FitC-Dextran is likely mainly due to passive diffusion in the initial phase. However, for 300 μg/mL of MMP-9, there was a significant increase in the release of FITC-Dextran at, for example, 24 hours. The inventors hypothesize that the increase in release for these gels is due to both passive diffusion and degradation of the GelMA+ gels by MMP-9.

The molecular weight of the FITC-Dextran used in the current study was 70 kDA. There are numerous known therapeutic molecules of similar size. For example, hyaluronic acid and bovine lactoferrin, used in corneal wound healing, have molecular weights greater than 60 kDa and 87 kDA respectively. Hyaluronic acid works by stimulating the migration, adhesion and proliferation of corneal epithelial cells, whereas bovine lactoferrin promotes corneal wound healing by upregulating certain cytokine production, such as IL-6 (Interleukin) and PGDF (platelet derived growth factor).

The second study evaluated the release of bovine lactoferrin from GelMA+ gels. The 30% (w/v) formulation GelMA+ was chosen because these gels had the best handling. The release trend for bovine lactoferrin from GelMA+ was very similar to that of the FITC-Dextran. At 0.5 and 12 hours, there were no differences between the control and the release media containing 100 μg/mL and 300 μg/mL of MMP9. These results suggest that the release of bovine lactoferrin from these gels is controlled mainly by passive diffusion between 0-12 hours. However, after 24 hours, the release was significantly higher for the MMP-9 solutions than the control. This would suggest that there may be a lag period before the MMP-9 can degrade the gels to release the matrix-bound bovine lactoferrin.

The GelMA+ gels in this study are degradable in the presence of MMP-9. MMP-9 and MMP-2 are enzymes which are released in response to a wounded state. This has been observed in wounds occurring in the eye, gut, skin and lungs. During the wounded state, the secretion and the activity of the MMPs are upregulated due to various cytokines and growth factors, including epidermal growth factor and hepatocyte growth factors (for example see: Pal-Ghosh S, Blanco T, Tadvalkar G, et al. Mmp9 Cleavage of the B4 Integrin Ectodomain Leads to Recurrent Epithelial Erosions in Mice. Journal of cell science 2011; 124:2666-75, the entire contents of which are incorporated herein by reference). MMPs have an important role at different stages of wound healing to regulate cell-cell and cell-matrix signalling, as well as tissue remodelling. A study with MMP-9 knock-out mice showed that wound closure was significantly delayed due to the absence of MMP-9.

However, while there was evidence that GelMA+ gels in this study released more FitC-Dextran and bovine lactoferrin in the presence of MMP-9, the MMP-9 concentration required for these observable differences are higher than physiologically reported values (for example see: Messmer E M, von Lindenfels V, Garbe A, Kampik A. Matrix Metalloproteinase 9 Testing in Dry Eye Disease Using a Commercially Available Point-of-Care Immunoassay. Ophthalmology 2016; 123:2300-8; and/or Hurst N G, Stocken D D, Wilson S, et al. Elevated Serum Matrix Metalloproteinase 9 (Mmp-9) Concentration Predicts the Presence of Colorectal Neoplasia in Symptomatic Patients. British journal of cancer 2007; 97:971-7, the entire contents of which are incorporated herein by reference). For instance, commercially available MMP-9 kits (InflammaDry; Rapid Pathogen Screening, Inc., Sarasota, Fla.) for dry eye require only a concentration above 40 ng/mL of MMP-9 in the tears for a positive detection (Messmer et al Ophthalmology 2016). The median MMP-9 concentration in serum for colorectal carcinoma was reported to be 443 ng/mL (Hurst et al British journal of cancer 2007). In other words, the amount of MMP-9 required for GelMA+ degradation in this study are an order of magnitude higher than those normally found in the body. That said, degradation effects of lower concentrations of MMP-9 may be seen over a longer period of time.

Overall, all GelMA+ gels in this study showed a slow release of both FitC-Dextran and bovine lactoferrin over at least 12 hours. The initial release phase was controlled primarily by passive diffusion. There was evidence that the release can be increased and extended by MMP-9, which degrades the gel to release the matrix-bound FitC-Dextran and bovine lactoferrin.

### Example 3—Gelatin Methacrylate as an Enzyme-Controlled Release Vehicle of Hyaluronic Acid for the Treatment of Recurrent Corneal Erosion

Recurrent corneal erosion (RCE) syndrome is a common clinical disease that is characterised by injured corneal epithelium and epithelial basement membrane. RCE is found to be 1 in 150 individuals, and additionally patients with lattice, granular, and macular dystrophy have shown an increased risk of obtaining RCE syndrome. Patients with RCE experience unpredictable and painful episodes, caused by the sporadic loss of superficial corneal epithelial cells (CEpCs). The erratic nature of RCE often leads to patient anxiety and can significantly impact one's productivity and quality of life. Although the true biological cause of RCE is unknown, it is speculated that the sporadic peeling of CEpCs occurs due to increased adhesive force between the corneal epithelium and tarsal conjunctiva. The poor cell attachment of the CEpCs to the underlying Bowman's membrane in RCE patients may aid the detachment process. Historically, patching (to prevent blinking) and ocular eye drops have been the standard treatment to relieve pain and improve corneal epithelial wound healing in RCE patients. Drug efficiency of ocular eye drops is less than 1% due to significant drug loss as the majority of active ingredients in the eye drops are washed away by the tear film. More recently, soft bandage contact lenses (BCLs) are increasingly being used, as this allows the use of vision during the healing process. Unfortunately, BCLs currently lack the biological molecules that are crucial to accelerate corneal epithelium wound closure. Controlled release of these therapeutically relevant molecules for RCE could effectively enhance and accelerate healing time for RCE patients.

When RCE occurs, an enhanced level of the matrix metalloproteinases (MMP) is observed. This causes deterioration within the corneal epithelium microenvironment and impacts cellular adhesion. The inventors hypothesized that the MMP-triggered controlled drug release from MMP responsive transparent hydrogel could be an effective strategy to improve the corneal epithelial wound healing. As the contact lens or ocular patches are placed directly over the cornea, they could serve as a reservoir for drug loading and subsequent delivery. Multiple studies have harnessed the potential of contact lenses to administer drugs for glaucoma treatment (for example, see: L. C. Bengani, K.-H. Hsu, S. Gause, and A. Chauhan, “Contact lenses as a platform for ocular drug delivery,” Expert Opinion on Drug Delivery, vol. 10, no. 11, pp. 1483-1496, 2013 Nov. 1, 2013, doi: 10.1517/17425247.2013.821462; and/or C. Mu, M. Shi, P. Liu, L. Chen, and G. Marriott, “Daylight-Mediated, Passive, and Sustained Release of the Glaucoma Drug Timolol from a Contact Lens,” (in eng), ACS Cent Sci, vol. 4, no. 12, pp. 1677-1687, 2018, doi: 10.1021/acscentsci.8b00641, the entire contents of which are incorporated herein by reference). For instance, Peng et al. showed that controlled delivery of timolol, a widely used glaucoma drug, could be 10 times more efficient compared to eye drops (for example, see: C.-C. Peng, M. T. Burke, B. E. Carbia, C. Plummer, and A. Chauhan, “Extended drug delivery by contact lenses for glaucoma therapy,” Journal of Controlled Release, vol. 162, no. 1, pp. 152-158, 2012 Aug. 20 2012 doi: https://doi.org/10.1016/j.conrel.2012.06:017, the entire contents of which are incorporated herein by reference). In another report, nanogel-poly(Hydroxyethyl)methacrylate (pHEMA) based contact lenses were used to encapsulate timolol, which could be released in a controlled fashion by using lysozyme as biological stimulus (for example, see: H. J. Kim, K. Zhang, L. Moore, and D. Ho, “Diamond nanogel-embedded contact lenses mediate lysozyme-dependent therapeutic release,” (in eng), ACS Nano, vol. 8, no. 3, pp. 2998-3005, Mar. 25 2014, doi: 10.1021/nn5002968, the entire contents of which are incorporated herein by reference). Effect of controlled release drugs on the treatment of RCE is less widely studied. Extended release of cyclosporine from silicon contact lenses has been demonstrated, which could potentially lower the MMP levels in RCE (for example, see: C. C. Peng and A. Chauhan, “Extended cyclosporine delivery by silicone-hydrogel contact lenses,” (in eng), J Control Release, vol. 154, no. 3, pp. 267-74, Sep. 25, 2011, doi: 10.1016/j.jconrel.2011.06.028, the entire contents of which are incorporated herein by reference). In a recent study, Mun et al. reported that the control release of cyclosporine from drug eluting composite pHEMA contact lens could effectively reduce the MMP levels in RCE microenvironment (for example, see: J. Mun, J. w. Mok, S. Jeong, S. Cho, C.-K. Joo, and S. K. Hahn, “Drug-eluting contact lens containing cyclosporine-loaded cholesterol-hyaluronate micelles for dry eye syndrome,” RSC Advances, 10.1039/C9RA02858G vol. 9, no. 29, pp. 16578-16585, 2019, doi: 10.1039/C9RA02858G, the entire contents of which are incorporated herein by reference). To the best of the inventors' knowledge, MMP responsive materials have not been studied as drug release agents for RCE syndrome. Such materials could provide a two-pronged approach to the treatment of RCE: (1) They can be used as MMP-triggered drug release material for improved wound healing; (2) MMP responsive hydrogels could potentially consume secreted MMPs, thereby lowering the deleterious effect of MMPs on the extracellular matrix (ECM).

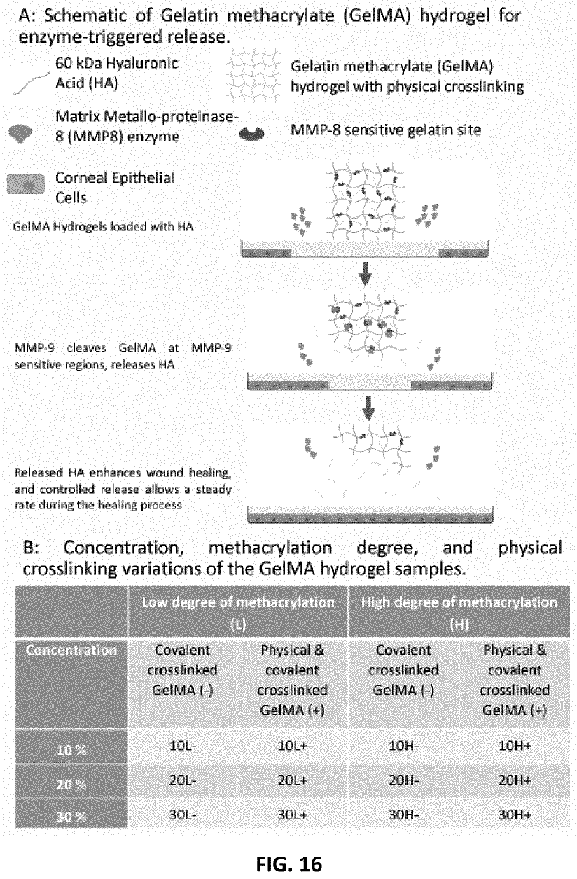

The objective of this study was to fabricate and characterize photocurable transparent gelatin methacrylate (GelMA) hydrogel as an MMP-triggered controlled release vehicle for hyaluronic acid (HA) delivery, and to test these molecules as viable drugs for enhancing corneal epithelial wound healing. GelMA based hydrogels are biocompatible, optically transparent, MMP cleavable biopolymer hydrogels that are widely used in tissue engineering and have some potential drug delivery uses (for example, see: K. Yue, G. Trujillo-de Santiago, M. M. Alvarez, A. Tamayol, N. Annabi, and A. Khademhosseini, “Synthesis, properties, and biomedical applications of gelatin methacryloyl (GelMA) hydrogels,” (in eng), Biomaterials, vol. 73, pp. 254-271, 2015, doi: 10.1016/j.biomaterials.2015.08.045; and/or Z. Luo, W. Sun, J. Fang, K. Lee, S. Li, Z. Gu, M. R. Dokmeci, and A. Khademhosseini, “Biodegradable Gelatin Methacryloyl Microneedles for Transdermal Drug Delivery,” (in eng), Adv Healthc Mater, vol. 8, no. 3, p. e1801054, February 2019, doi: 10.1002/adhm.201801054, the entire contents of which are incorporated herein by reference). We have previously used GelMA hydrogels films as cell carrier and transplantation device for corneal endothelial cells (for example, see: Rizwan et al. Biomaterials 2017). HA is responsible for numerous functions in the human body including a critical role in the ECM for cell migration and proliferation. HA has demonstrated the ability to improve rabbit corneal epithelial wound healing in vivo in n-heptanol, iodine vapour and mechanical scraping models. The inventors used 70 kDa Fluorescein Isothiocyanate (FITC)-dextran as a model drug molecule to study the effect of concentration of GelMA prepolymer, methacrylation degree, and mode of crosslinking (covalent crosslinking vs. sequential physical+ covalent crosslinking) on the controlled release profile of model drug (FIG. 16A, 16B). The rabbit corneal epithelial cell (RCEpC) and human corneal epithelial cell (HCEpC) migration in response to the different concentration of HA was investigated by using in vitro wound healing assays.

### Synthesis of Gelatin Methacrylate

GelMA hydrogels used for the controlled release and drug loading studies was made from type A gelatin (Sigma-Aldrich). Type A gelatin was mixed in phosphate-buffered saline (PBS; Fisher Scientific) to make a 10%, 20%, or 30% (w/v) solution at 50° C. for one hour or until fully dissolved. Methacrylic anhydride (MA; Sigma-Aldrich) was then added to the gelatin solution at a rate of ˜0.5 ml/min. Two types of GelMA with different degrees of methacrylation were prepared for this study: GelMA hydrogels with high (about 90%) and low (about 50%) degrees of methacrylation where 10 mL and 125 μL of MA solution were added respectively. Once the respective amounts of MA were added to the gelatin, the solution was stirred to react for one hour at 50-60° C. The reaction was stopped by adding a 1× dilution of ˜40° C. PBS. Impurities and low molecular weight GelMA were removed from the reacted solution via centrifugation using a Sorvall ST 16R centrifuge (Thermo Scientific) at 5000 RPM for 5 minutes. The GelMA supernatant was collected and dialyzed in 12¬14 kDa dialysis tubing (Spectra/Por®) in deionized water at ˜40° C. The deionized water was changed every 2 hours in an 8-hour period for the first two days and daily thereafter for 5-7 days. The dialyzed solution was freeze-dried using a FreeZone 1 L freeze dryer (LABCONCO) at −80° C. for a week to remove the water content. The lyophilized GelMA powder was stored at −20 or −80° C. until further use.

Fluorescein Isothiocyanate (FITC)-Dextran Loaded GelMA Fabrication

IRGACURE 2959 Photo Initiator (Sigma-Aldrich) was dissolved in PBS at 60° C. for one hour to make a 0.5% solution. Lyophilized GelMA powder was dissolved in the solution at 60° C. for 2 days to ensure the powder was completely solubilized. FITC-dextran (4 kDa and 70 kDa; Sigma-Aldrich) was added to the GelMA solution to achieve a 5 mg/ml FITC-dextran concentration. The solution was vortexed to ensure homogeneity and centrifuged at 5000 RPM for 5 minutes to remove bubbles. To reverse any physical gelling that may have occurred during the centrifugation step the solution was then heated to 60° C. in an oven. Approximately 200 μL of the GelMA and FITC-dextran solution was pipetted into a transparent holding chamber composed of glass slides of dimensions 22×˜15×0.5 mm. The chamber filled with GelMA hydrogel precursor solution was exposed to ultra-violet light from the HTBX UVLED curing oven (EIGHT-LED) at various intensities for 300 s. The solid gel was removed from the chamber and cut into disks.

Controlled Release of FITC-Dextran Via Enzymatic Degradation of GelMA

To characterize their enzymatic degradation and resulting release profile, 6 mm diameter GelMA disks were placed in a 24-well plate and incubated at 37° C. To remove any surface adsorbed molecules, the disks were washed with 1 ml of PBS for 30 minutes. After washing the disks were incubated with 1 μg/ml MMP-8 (GIBCO, Thermofisher) or 250 ng/ml MMP-9 (GIBCO, Thermofisher) in PBS. The enzyme solutions were collected and replaced at 2, 4, 6, 8, 10, 12 hours and subsequently every 24 hours for 7 days. At the end of the 7 days, 200 μg/ml of the respective enzyme solution were added to the samples for overnight incubation at 37° C. to completely degrade the GelMA hydrogels. At the previously defined time points, the collected enzyme solutions were placed in a 48-well plate and stored in −80° C. until ready for quantification. 100 μl of sample solution collected at each time point was transferred to a black 96-well plate and fluorescence was quantified using a plate reader, at a fluorescence excitation of 490 nm and emission of 520 nm. The accumulative release was calculated in Excel using a FITC-dextran calibration curve and graphed using GraphPad Prism 6.

Cell Culture and HA Assisted CEpC Wound Healing Assays

Primary rabbit corneal epithelial cells (Cell Biologics Inc.) were cultured using a T-25 flask in epithelial cell media supplemented with insulin-transferrin-selenium, epidermal growth factor, L-glutamine, antibiotic-antimycotic solution and fetal bovine serum (Cell Biologics Inc.). Once confluence was reached, the RCEpCs were briefly rinsed with PBS and passaged with trypsin-EDTA (GIBCO, Thermo Scientific). The cells were then sub-cultured on a 24-well plate surface pre-coated with a gelatin-based coating (Cell Biologics Inc.) containing a thin, punched out poly-dimethyl siloxane (PDMS) stencil adhered to the surface. The media was changed daily until a cell monolayer was formed. The PDMS disk stencil was then carefully removed from the surface. The well was then rinsed with PBS and replaced with epithelial cell media. Images were taken daily to observe the closure of the wound and graphs were generated using GraphPad Prism 6.

HCEpCs were cultured in a T-25 flask with keratinocyte serum-free media supplemented with keratinocyte growth serum and penicillin/streptomycin (KM; Sciencell) that was changed every other day. The SV-40 immortalized HCEpCs were generously donated from a collaborator (for example, see: F. Li, M. Griffith, Z. Li, S. Tanodekaew, H. Sheardown, M. Hakim, and D. J. Carlsson, “Recruitment of multiple cell lines by collagen-synthetic copolymer matrices in corneal regeneration,” Biomaterials, vol. 26, no. 16, pp. 3093-3104, 2005 Jun. 1, doi: https://doi.org/10.1016/j.biomaterials.2004.07.063; and/or M. Griffith, R. Osborne, R. Munger, X. Xiong, C. J. Doillon, N. L. C. Laycock, M. Hakim, Y. Song, and M. A. Watsky, “Functional Human Corneal Equivalents Constructed from Cell Lines,” Science, vol. 286, no. 5447, pp. 2169-2172, 1999, doi: 10.1126/science.286.5447.2169, the entire contents of which are incorporated herein by reference). Once confluence was reached, the HCEpCs were passaged in 2 ml of TrypLE Express (GIBCO, Life Technologies, Thermo Scientific) and subcultured for the wound assay as described previously. The HCEpCs were cultured with KM until a monolayer had formed. Wound assays were conducted as described above for RCEpCs. The well was then rinsed with PBS and replaced with KM. Images were taken daily to observe the closure of the wound and graphs were generated using GraphPad Prism 6.

To validate the wound assays 200 kDa hyaluronic acid (151-300 kDa HA; Lifecore Biomedicals) were added to the media of both RCEpC and HCEpC PDMS wound assays. Once the wound assays were validated, the efficacy of medium sized 60 kDa HA (66-99 kDa; Lifecore Biomedicals) was investigated. Various concentrations of 60 kDa HA were dissolved in KM and added as a bolus dosage to HCEpC wound assays. The media was changed daily until full closure of the wound. Images were taken daily to observe the closure of the wound and graphs were generated using GraphPad Prism 6.

Controlled Release of HA from GelMA Hydrogels and ELISA Detection

To test the bioactivity of HA released from GelMA samples, 8 mm GelMA patches containing 0, 150, 250, 550, 750 μg of 60 kDa HA were fabricated similarly to the FITC-dextran hydrogels and placed in a 24 well-plate. A 250 ng/ml MMP-9 solution of KM was added to each well and replaced at 12, 36, 60 hours and every 24 hours afterwards for 5 days. The replaced media was collected in another 24-well plate and stored in −80° C. A HCEpCs PDMS wound assay was prepared as described prior. After the PDMS removal and the PBS washing step, plain KM was added for 24 hours. The media was then replaced with the subsequent collected and stored HA containing media samples until the wound was healed. Images were taken every 12-24 hours to monitor the rate of wound healing and curves were created using GraphPad Prism 6.

To detect and quantify the amount of HA released, an ELISA was conducted. Either samples, standards, or diluent was added to the wells with 50 μl of the working detector solution except for the black control wells. The plate was covered with a plate seal and incubated for one hour at 37° C. 100 μl of each sample was then transferred to the detection plate and gently mixed. The detection plate was covered and incubated at 4° C. for 30 minutes. The solution was then removed from the plate and the wells were rinsed with 1× wash buffer. Once sufficiently washed, 100 μL of the working enzyme was added to each well and incubated at 37° C. for 30 minutes, covered with a plate seal. The solution was removed, and each well was rinsed with wash buffer. 100 μl of working substrate solution was added to each well, and the plate was incubated at room temperature in a dark environment. The absorbance at 405 nm was measured at 15 minutes and 30 minutes and the reaction was stopped by adding 50 μl of stopping solution to each well. A best-fit curve was generated using non-linear regression analysis with GraphPad Prism 6 and a sigmoidal dose response-variable slope curve (four-parameter) analysis was used to determine the concentration of each well as described in the Echelon procedure.

Statistics

Statistical evaluation was conducted using GraphPad Prism 6 software using a one-way analysis of variance (ANOVA) for single comparisons and non-parametric analysis, while two-way ANOVA was used for multiple comparisons such as the wound assays. The number of experimental replicas are stated in each figure.

Results and Discussion

Effect of molecular size on enzyme-triggered drug release behavior

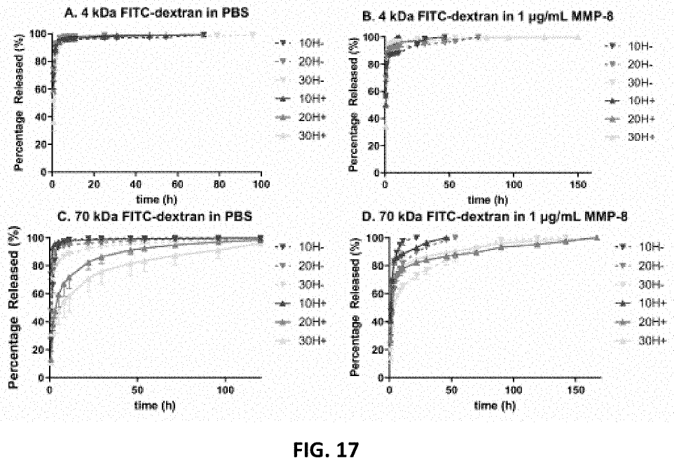

GelMA patches with high degrees of methacrylation were loaded with either 4 or 70 kDa FITC-dextran and incubated in PBS or 1 μg/ml MMP-8 solution to determine the release characteristic of different sized molecules. MMP-8 was used in the initial validation experiments to represent an advanced case of recurrent corneal erosion (RCE), as it is also upregulated in RCE and is known to have a faster degradation rate of gelatin then MMP-9 (for example, see: N. R. Alan Barrett, J. Woessner, Handbook of Proteolytic Enzymes Third ed. Academic Press (in English), 2012; and/or B. Ratnikov, E. Deryugina, J. Leng, G. Marchenko, D. Dembrow, and A. Strongin, “Determination of matrix metalloproteinase activity using biotinylated gelatin,” (in eng), Anal Biochem, vol. 286, no. 1, pp. 149-55, Nov. 1, 2000, doi: 10.1006/abio.2000.4798, the entire contents of which are incorporated herein by reference). It is also a well-studied enzyme for gelatin hydrogel biodegradation and could facilitate direct comparison of the data to the existing literature. GelMA hydrogels with high degrees of methacrylation were used due to the effect of additional crosslinking reducing the pore size of the hydrogel, and allowed us to determine if GelMA hydrogels can encapsulate low and high molecular weight molecules (for example, see: U. Eckhard, P. F. Huesgen, O. Schilling, C. L. Bellac, G. S. Butler, J. H. Cox, A. Dufour, V. Goebeler, R. Kappelhoff, U. A. D. Keller, T. Klein, P. F. Lange, G. Marino, C. J. Morrison, A. Prudova, D. Rodriguez, A. E. Starr, Y. Wang, and C. M. Overall, “Active site specificity profiling of the matrix metalloproteinase family: Proteomic identification of 4300 cleavage sites by nine MMPs explored with structural and synthetic peptide cleavage analyses,” (in eng), Matrix Biol, vol. 49, pp. 37-60, January 2016, doi: 10.1016/j.matbio.2015.09.003, the entire contents of which are incorporated herein by reference). The percentage of FITC-dextran released was calculated to demonstrate the completion of the controlled release. The 4 kDa FITC-dextran completely eluted from the hydrogel within the first 10 hours, implying a diffusion-limited release profile (FIGS. 17A and 17B). This revealed that the drug freely permeated without obstruction through the gel despite the smaller pore size from the additional crosslinking. However, the 70 kDa FITC-dextran demonstrated a concentration and crosslinking-dependent release profile (FIGS. 17C and 17D), implying an enzyme-mediated release. This suggests that longer release profiles may be seen with larger payloads. However, modifications could be made to further tune the system to achieve a longer sustained release profile for smaller payloads.

GelMA Concentration and Methacrylation Degree Effect on the Controlled Release of FITC-Dextran Model Drug from Hydrogels

The release characteristic of several formulations of GelMA were investigated in order to determine optimal controlled release response of a model drug, 70 kDa FITC-dextran. Three different hydrogel concentrations (10%, 20%, 30%) with either high (H) or low (L) methacrylation degrees were crosslinked under 600 mW/cm2 for 300 s. The effect of sequentially-crosslinked GelMA (GelMA+) (see Rizwan Biomaterials 2017) was also investigated to compare the release profile to that of regular GelMA (GelMA−). This resulted in a set of GelMA hydrogels with different characteristics, as summarized in FIG. 16B.

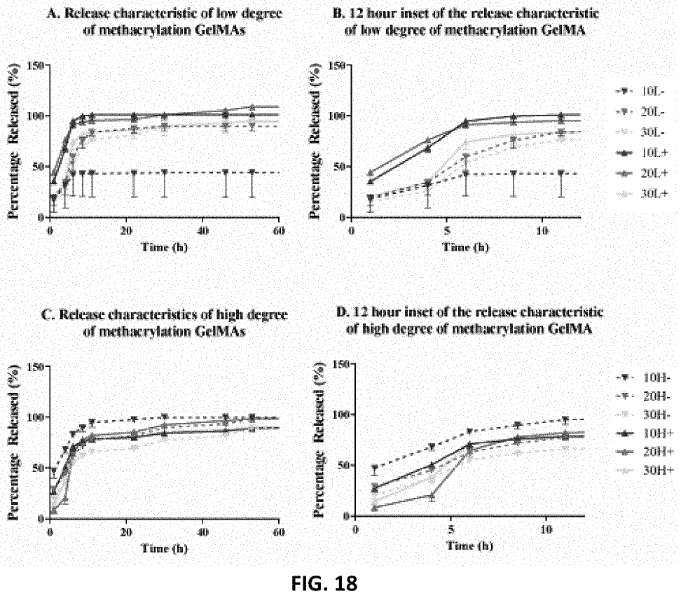

The release kinetics of the 70 kDa FITC-dextran loaded GelMA (and GelMA+) samples were observed over the course of 5-7 days in the presence of a 1 μg/ml MMP-8 solution. All low degrees of methacrylation GelMA/GelMA+ samples fully degraded within three days (FIG. 18A) and experienced an initial burst release of FITC-dextran within the first 6 hours (FIG. 18B). The high degree of methacrylation GelMA/GelMA+ samples demonstrated a broad range of degradation time when exposed to MMP-8 (FIG. 18C). However, they also experienced a reduced burst release in the first 6 hours (FIG. 18D). The increased methacrylation degree provided more crosslinking sites between the methacrylated lysine residues, slowing the breakdown of the gel within the MMP-8 solution.

The effect of GelMA+ on the control release of FITC-dextran was also investigated. It is noted that all GelMA+ samples continued to slowly release FITC-dextran even at the last time point of the experiment, compared to the GelMA−(GelMA) samples which stopped release FITC-dextran at 70 hours for 10H− and 20H−. The three different GelMA+ concentrations outperformed the 30H-sample that stopped releasing at 142 hours. Similarly, there was improved structural support provided by additional physical crosslinking at 4° C. (see Rizwan Biomaterials 2017). The reduced pore size created a membrane that was difficult for the enzyme to permeate through, resulting in a reduced burst release. This effect could happen in the GelMA+ samples due to the increased crosslinking density reducing the pore size and extending the enzymatic cleavage time.

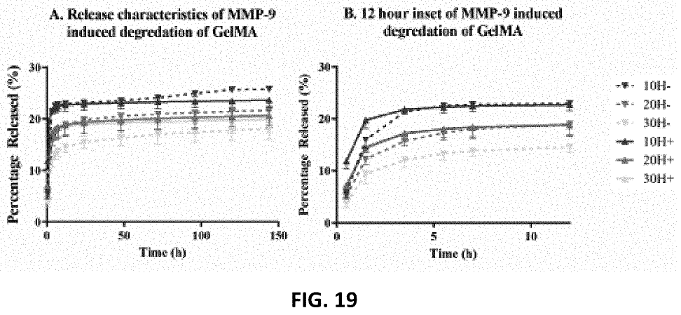

The degradation of the high degree of methacrylation GelMA/GelMA+ was also examined using a 250 ng/ml MMP-9. As MMP-9 is upregulated in patients of recurrent corneal erosion, it was necessary to investigate the controlled release profile of GelMA/GelMA+ degraded by MMP-9. Higher concentration GelMA/GelMA+ samples resulted in a lower burst release of FITC-dextran in the MMP-9 solution. This is shown by a significant decrease in the release between the 10H− and 20H− patches from 3.5 to 7 hours (p<0.03; FIG. 19A), and between the 10H− and 30H− patches from the 1.5 hours onwards (p<0.0001; FIG. 19B). The GelMA+ samples did not further reduce the burst release rate of MMP-9 as it had with the MMP-8. Comparing FIG. 19A to FIG. 18C, lower FITC-dextran was released from the FITC-dextran-loaded GelMA samples in 250 ng/ml of MMP-9 (FIG. 19A), compared to in 1 μg/ml of MMP-8 (FIG. 18C). As all physical parameters were identical in both experiments, the outlying differences, including the reduced burst release and released amount would be related to the enzyme types and the enzyme concentration. This was further verified in another experiment when the release of FITC-dextran was compared in the presence of 1 μg/ml of MMP-8 and 1 μg/ml of MMP-9 in the presence of the same concentration of the MMP solution, lower amount of FITC-dextran was released in the presence of MMP-9. The observed results could be due to the different mechanisms of peptide cleavage between MMP-9 and MMP-8.

In brief, the release profile of the FITC-dextran model drug was tuned from a few hours to several days by studying the effects of the additional physical crosslinking strategy, adjustment of the methacrylation degree, and the concentration of GelMA/GelMA+ on the control release. A tunable controlled release profile could be developed by altering the porosity, crosslinking site density, and permeability by varying the previous parameters. The additional physical crosslinking step in “+” samples provided extra structural support allowing for a longer enzymatic cleavage time. The sequential crosslinking also reduced the pore size, creating an environment that was more difficult for the drug to permeate through, thereby causing a lower burst release. A higher methacrylation degree increased the number of crosslinking sites, resulting in a more crosslinked gel. The higher concentrations of GelMA/GelMA+ demonstrated a denser polymer matrix inside the gel, which significantly reduced the rate of diffusion of FITC-dextran to the external environment. With this analysis of the hydrogel properties, we are able to tune the release profile over an extended period of days. The release profile could be extended even longer with further tuning.

Validation of the Efficacy of HA to Promote Wound Healing in Corneal Epithelial Wound Healing Assays

HA is a naturally occurring polymer within the skin known to induce in vitro healing of HEpCs. An effective wound assay was determined to demonstrate the efficacy of HA as a wound healing drug for CEpCs. Initially, concentrations of 0.45 and 0.75 mg/ml of HA were added as bolus doses to mimic literature. Previous literature on HA for corneal epithelial cells used either oligo-HA (<10 kDa) or native high molecular weight. As 4 kDa FITC-Dextran eluted in the enzyme-mediated GelMA release previously, 200 kDa HA was used for preliminary wound assays to validate the wound assay.

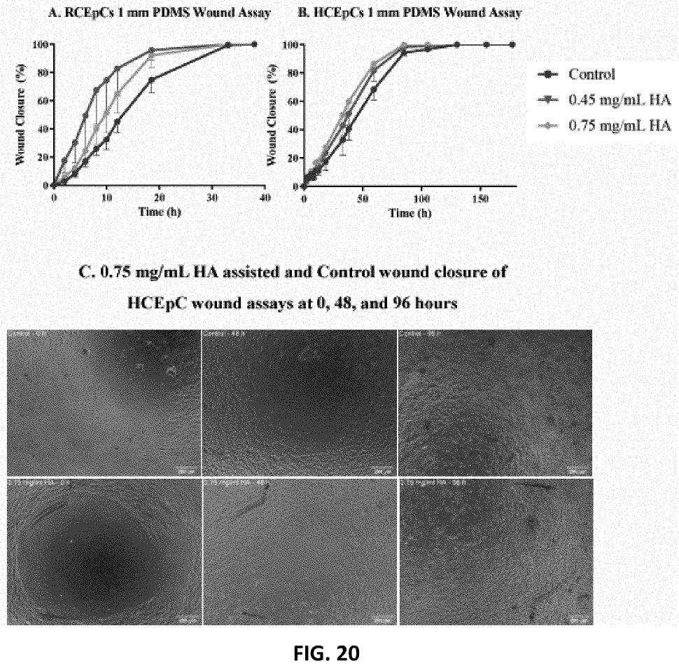

To verify if the wound assay could provide a consistent initial wound area to two different corneal epithelial cell types, 1 mm diameter PDMS wound assays were performed with both rabbit corneal epithelial cells (RCEpCs; FIG. 20A) and human corneal epithelial cells (HCEpCs; FIG. 20B). Primary RCEpCs were initially used as they would behave similar to future in vivo rabbit studies. Healing of the wound varied between the two cell-types, with the primary RCEpC wound healing within 2 days and the HCEpC wound healing in 7 days (FIG. 20C). The RCEpC wound assay showed that the 0.45 mg/ml HA sample was consistently faster than the 0.75 mg/ml of HA throughout the experiment, boasting a 149% (36.4%±14.6% wound closure in 0.45 mg/ml HA) improvement relative to the control (8.3%±2.5% wound closure) in wound healing at 4 hours. The improvement was narrowed to 21% (95.9%±6.9% wound closure) at 18.5 hours, compared to the control (74.9%±9.0%). However, the higher concentration of HA was less effective on the RCEpCs PDMS-stencil wound assay, demonstrating an improvement of 35% (64.9%±13.6% wound closure in 0.75 mg/ml of HA) compared to control (45.2%±7.8%) at 12 hours. The HCEpCs wound assay revealed that the 0.75 mg/ml of HA offered significant healing with a 58% (27.5%±7.1% wound closure in 0.75 mg/ml of HA) improvement at 18.5 hours that narrowed to 26% (86.5%±4.2% wound closure) at 59.5 hours compared to the control (17.4%±5.7% and 68.6%±7.4% wound closure, respectively, at 18.5 hours and 59.5 hours). The 0.45 mg/ml HA sample demonstrated similar wound healing, with a 31% (42.8%±7.2% wound closure) improvement, compared to the control (32.8%±11% wound closure) at 33 hours that narrowed to 19% improvement (81.8%±7.6% wound closure) at 59.5 hours, compared to the control (68.6%±7.4% wound closure). HA was confirmed to be a potential drug due to its consistency among cell types, rapid ability to heal wounds, low minimum effective concentration, and cost efficiency.

Effect of Using 60 kDa HA on the Rate of Corneal Epithelial Wound Healing

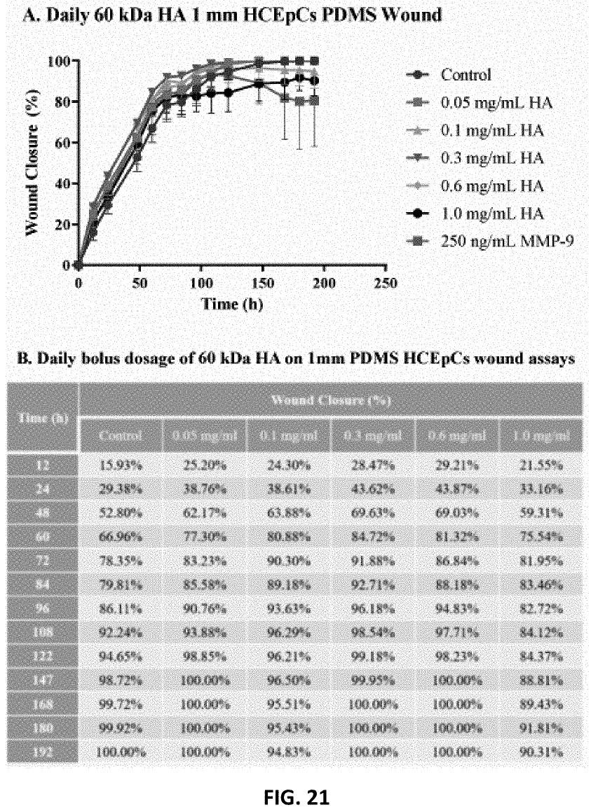

Most literature assesses the effects of native high molecular weight HA and oligomer HA on epithelial wound healing. To obtain an ideal MMP-9 controlled release profile, the influence of mid-size 60 kDa HA on corneal wound healing was investigated. PDMS-stencil wound assays were conducted using 0.05, 0.1, 0.3, 0.6, or 1.0 mg/ml of 60 kDa HA treatments daily (FIG. 21A). A 250 ng/ml MMP-9 sample was included to observe the effects of the enzyme on wound healing, similar to an in vivo environment. It was shown that the 0.3, 0.6, and 0.05 mg/ml HA supplemented wounds healed rapidly, whereas the other HA samples slowed wound healing compared to the control, as summarized in FIG. 21B. The 250 ng/ml MMP-9 treated sample initially had no effect on the wound healing, but caused the wound to expand by 120 hours. This decrease in wound healing was most significant at 168, 180 and 192 hours with a deterioration relative to the control (99.7%±0.6%, 99.9%±0.2%, and 100%±0.0% wound closure, respectively) for each time point of ˜3.4% (96.3%±20.5% wound closure), −3.0% (96.9%±23.6% wound closure) and −0.9% (99.1%±22.4% wound closure), respectively. The results validate the use of a mid-size HA molecule in accelerating HCEpCs wound healing and suggested a therapeutic range between 0.1-′0.6 mg/ml for HCEpCs.

Controlled Release of HA from GelMA and ELISA Determination of HA

The data collected in the previous FITC-dextran controlled release experiments and the HA supplemented PDMS stencil wound assays were combined to investigate the efficacy of HA released from a GelMA patch on wound healing. A 10H− GelMA patch of 8 mm diameter was used instead of the previously tested 6 mm patches to increase the amount of 60 kDa HA loaded into the hydrogels.

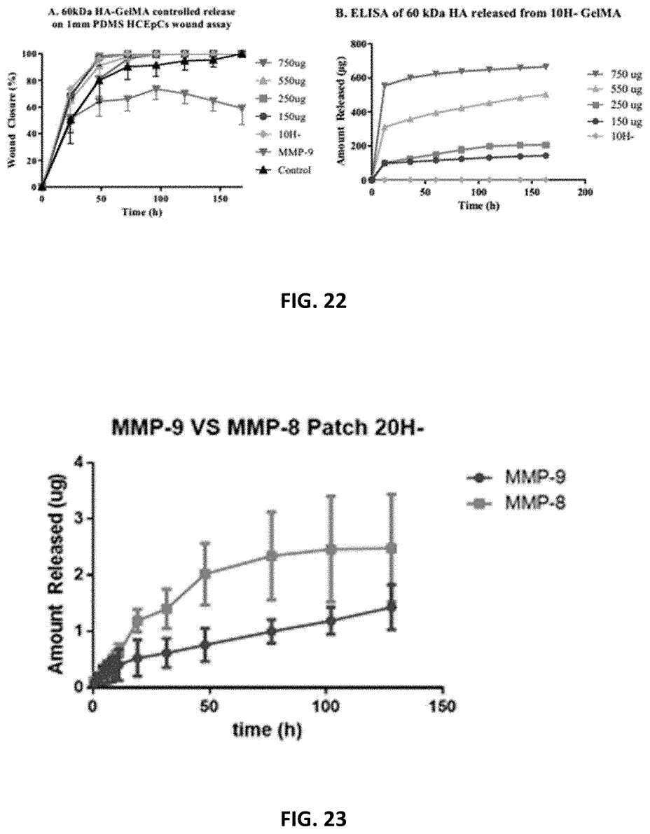

The bioactivity of 60 kDa HA loaded in a GelMA patch is essential for the intended application of the hydrogels as a method for corneal wound healing. HA binds to cell surface receptors such as CD44. CD44 signaling is believed to mediate corneal wound healing. However, chemical modification of HA is known to reduce its CD44 binding ability. Therefore, instead of chemically modifying HA to fabricate HA hydrogels, we delivered unmodified HA by using MMP-responsive GelMA hydrogels to preserve its bioactivity. Every GelMA patch provided an accelerated wound closure (FIG. 22A). The 150 μg and 250 μg loaded GelMA patches displayed an accelerated wound healing at 24 hours of 28% (68.7%±17.4% wound closure) and 26% (67.9%±13.1% wound closure), and 48 hours mark of 16% (96.8%±5.8% wound closure) and 18% (98.3%±3.5% wound closure) improvement, compared to the control (50.5%±17.8% and 79.9%±16.7% wound closure at 24 and 48 hours, respectively). In the presence of 750 μg loaded-GelMA sample, the wound healing was slower than that of the lower HA loaded-GelMA samples and 10H− control GelMA patch without any HA (10H−), it still showed improved wound closure of 21% (48.8%±22.6% wound closure) after 24 hours compared to control (50.5%±17.8% wound closure). Interestingly, the 10H-sample showed a improvement of 37% (73.7%±19.7% wound closure) and 16% (96.4%±4.1% wound closure) compared to the control (50.5%±17.8% and 79.9%±16.7% wound closure) at 24 and 48 hours respectively. The results suggested that the degraded 10H− patch improved the wound healing rate of HCEpCs. GelMA hydrogels are based on gelatin, a biomolecule known to aid wound healing of in vitro cell culture. Gelatin-based coatings containing a relatively low concentration of gelatin ˜0.1% are a common supplement to aid in cell adhesion. The gelatin degraded by MMP-9 could attach to the well plate surface allowing for rapid migration over the surface. The wounds supplemented with the 550 μg and 750 μg HA-loaded GelMA patch closed later than the lower HA-loaded GelMA and the 10H− patch suggesting a released concentration may be above the minimum toxic concentration, slowing the wound healing.

To test and quantify the controlled release of HA from GelMA patches, an ELISA was conducted to confirm that the minimum effective concentration was obtained. Based on prior controlled release experiments, 100 μl of media that was used for the bioactivity assay was diluted to the appropriate linear range of the calibration curve given by the ELISA kit (50-1600 ng/ml). Based on the previous FITC-dextran controlled release experiments, the dilution was estimated as an 85% burst release and a 1.5% release rate over seven days. The 750 μg, 550 μg, 250 μg, and 150 μg had a burst release of 83%, 62%, 49%, and 68%, respectively, within the first 12 hours. After this, the 150, 250, and 550 μg samples exhibited a 5% daily release rate, and the 750 μg samples a 2% daily release rate (FIG. 7B). The 10H− samples were fully degraded at the final time point. The percentages of the HA that was loaded into the 10H− compared to the theoretical value for the 750, 550, 250, and 150 μg samples were found to be 96%, 90%, 93%, and 83%, respectively. The reduced loading into the 750 μg sample could be due to a saturation of the solution, as the 750 μg samples contained particles of 60 kDa HA on the wound assay that were not fully dissolved potentially residing on the outside of the 10H− patch. No such residue was found on 10H− samples loaded with a lower amount of 60 kDa HA.

### CONCLUSION

GelMA/GelMA+ was investigated as an enzyme-triggered controlled drug release material for the treatment of recurrent corneal erosion syndrome. A tunable profile was determined by altering the porosity, crosslinking density, and permeability through the matrix by sequential crosslinking, adjustment of the methacrylation degree, concentration of GelMA and UV exposure time. The effect of the molecular size of the model drug molecule on the MMP triggered release profile was investigated. A 70 kDa FITC-dextran molecule demonstrated a release profile dependent on the concentration and sequential crosslinking of the GelMA/GelMA+. HA demonstrated an increased wound closure in wound assay models at the 0.45 mg/mL and 0.75 mg/mL concentrations. A 60 kDa HA in the therapeutic range between 0.1-0.6 mg/mL increased the wound healing rate by 40% in the first 24 hours, and by 24% in the 60 hours. A controlled release of the 60 kDa HA from a 10H− GelMA patch demonstrated promising results with the 150 μg and 250 μg HA loaded gels improving the wound closure by 28% (68.7%±17.4% wound closure) and 26% (67.9%±13.1% wound closure) at the 24-hour mark compared to the control, respectively. The results validated the GelMA/GelMA+ patches as a viable vehicle for controlled delivery of 60 kDa HA without disturbing the bioactivity of the loaded molecule. Overall, we demonstrated that a transparent, tunable GelMA/GelMA+ material could be suitable material for drug delivery in RCE syndrome.

### Example 4—Effect of Enzyme Type (MMP-8 VS MMP-9)

MMP-8 and MMP-9 cleave gelatin differently. In order to differentiate the release profile between MMP-8 and MMP-9, the enzymes were compared. An experiment degrading a 6 mm pure GelMA patch was performed over 5 days using 1 μg/mL of either MMP-8 or MMP-9 (FIG. 23).

An interesting phenomenon occurred where the GelMA patches degraded in MMP-8 (FIG. 23) released 70 kDa FITC-dextran faster than ones in MMP-9. Since RCE is primarily caused by MMP-9 enzyme, future experiments focused on this enzyme, however, as several MMP enzymes are present in the tear film during RCE, it is advised that the effect of each enzyme at physiological concentrations on GelMA degradation is investigated.

Reflecting on the controlled release studies, despite the quenching of FITC-dextran, the model molecule provided an accurate prediction of the release profile for the molecule selected. Highly methacrylated in particular demonstrated a large tunable profile within the 5-7 day range based on concentration of GelMA dissolved in PBS solution containing Pl.

All references cited in this document are incorporated herein by reference in their entirety.

The above-described embodiments are intended to be examples only. Alterations, modifications and variations can be effected to the particular embodiments by those of skill in the art without departing from the scope of the disclosure, which is defined solely by the claims appended hereto.

1. A hydrogel system for delivering a payload comprising:
a hydrogel comprising a crosslinkable polymer, such as a chemically-modified biopolymer, for example, chemically-modified gelatin, the hydrogel formed by a method comprising sequential physical and chemical crosslinking steps; and
a payload.

2. The hydrogel system of claim 1, wherein the chemically-modified gelatin comprises methacrylated gelatin, acrylated gelatin, thiolated gelatin or a combination thereof.

3. The hydrogel system of claim 1 or 2, wherein the chemically-modified gelatin comprises methacrylated gelatin.

4. The hydrogel system of any preceding claim, wherein the physical crosslinking step comprises incubating a solution comprising the crosslinkable polymer under suitable conditions and for a sufficient period of time to permit physical crosslinking of at least a portion of the crosslinkable polymer.

5. The hydrogel system of claim 4, wherein the solution comprises between about 1%-35% (w/v) of the crosslinkable polymer in a suitable diluent, e.g., about 1%-about 5%, about 1%-about 10%, about 5%-about 30%, about 10%-about 35%, about 10%-about 30%, about 10%-about 20%, about 20%-about 30%, about 25%-about 35% or about 1%, about 5%, about 10%, about 15%, about 20%, about 25%, about 30%, or about 35%.

6. The hydrogel system of claim 5, wherein the solution is an aqueous solution.

7. The hydrogel system of claim 5 or 6, wherein the diluent is phosphate buffered saline (PBS).

8. The hydrogel system of any preceding claim, wherein the incubation period is at least 1 hour.

9. The hydrogel system of any preceding claim, wherein the incubation period is between about 15 minutes to about 3 hours, between about 30 minutes to about 2 hours, between about 45 minutes to about 1.5 hours, or about 15 minutes, about 30 minutes, about 45 minute, about 1 hour, about 1.5 hours, about 2 hours, or about 3 hours.

10. The hydrogel system of any preceding claim, wherein the physical crosslinking step comprises incubation at a temperature between about 1-about 16° C., between about 2-about 15° C., between about 2-about 10° C., between about 3-about 8° C., between about 4-about 6° C., between about 3-about 5° C. or about 2° C., about 3° C., about 4° C., about 5° C., about 6° C., about 7° C., about 8° C., about 10° C., about 12° C., about 14° C., or about 16° C.

11. The hydrogel system of any one of the preceding claims, wherein the chemical crosslinking step is a thermal process.

12. The hydrogel system of any one of the preceding claims, wherein the chemical crosslinking step comprises UV irradiation.

13. The hydrogel system of claim 12, wherein the UV irradiation takes place in the presence of a photoinitiator.

14. The hydrogel system of claim 12, wherein photoinitiator is selected from the group consisting of 2-hydroxy-4′-(2-hydroxyethoxy)-2-methylpropiophenone (Irgacure 2959, or IC2959); lithium phenyl-2,4,6-trimethylbenzoylphosphinate (LAP); 2,2′-azobis[2-methyl-n-(2-hydroxyethyl)propionamide] (VA-086); or 2′,4′,5′,7′-tetrabromofluorescein disodium salt (Eosin Y).

15. The hydrogel system of claim 12, wherein the chemical crosslinking step comprises UV irradiation for between about 10 seconds to about 30 minutes, for example between about 10 seconds to about 30 seconds, between about 30 seconds to about 90 seconds, between about 10 seconds to about 1 minute, between about 1 minute to about 5 minutes, between about 1 minute to about 2 minutes, between about 2 minutes to about 5 minutes, between about 5 minutes to about 10 minutes, between about 10 minutes to about 20 minutes, or about 10 seconds, 30 seconds, 60 seconds, 90 seconds, 2 minutes, 3 minutes, 4 minutes, 5 minutes, 10 minutes, 15 minutes, 20 minutes, or 30 minutes.

16. The hydrogel system of claim 12, wherein the chemical cross-linking step comprises UV irradiation with between about 360-480 nm, such as between about 360-450 nm, between about 380-480 nm, between about 400-450 nm, between about 360-400 nm, or about 360 nm, 370 nm, 380 nm, 390 nm, 400 nm, 410 nm, 420 nm, 430 nm, 440 nm, 450 nm, 460 nm, 470 nm, or 480 nm.

17. The hydrogel system of any one of the preceding claims, wherein the hydrogel comprises a polymer consisting of gelatin methacrylate.

18. The hydrogel system of any previous claim, wherein gelatin methacrylate is the sole polymer forming the matrix of the hydrogel.

19. The hydrogel system of any one of claims 1 to 17, wherein the hydrogel comprises one or more additional polymers.

20. The hydrogel system of claim 19, wherein the one or more additional polymers is selected from hydrogel polymers, carboxybetaine methacrylate (CBMA), Alginate hydrogel, poly(hydroxylethylmethacrylate) (HEMA), Collagen derivatives, Poly lactic glycolic acid (PLGA), or Acrylamide gels.

21. The hydrogel system of any one of the preceding claims, wherein at least a portion of the payload is encapsulated within a matrix formed by the hydrogel.

22. The hydrogel system of claim 21, wherein at least about 50-99% of the payload is encapsulated within a matrix formed by the hydrogel, for example, at least about 50%, 60%, 70%, 75%, 80%, 85%, 90%, 95%, 97%, 98%, or 99%.

23. The hydrogel system of any one of the preceding claims, wherein a portion of the payload is associated with a surface of the hydrogel.

24. The hydrogel system of any one of the preceding claims, wherein the payload is a therapeutic agent, preventative agent, marker, cell, or the aforementioned members encapsulated in another delivery vehicle such as a nanoparticle or liposome.

25. The hydrogel system of any one of the preceding claims, wherein the payload is a small molecule or biologic molecule.

26. The hydrogel system of any one of the preceding claims, wherein the payload is sufficiently large such that all or a portion of the payload is retained within a matrix formed by the hydrogel until degradation of the hydrogel occurs.

27. The hydrogel system of any one of the preceding claims, wherein the molecular weight of the payload is less than about 100 kDa, less than about 70 kDa, less than about 50 kDa, less than about 30 kDa, less than about 15 kDa, less than about 10 kDa, less than about 4 kDa, or less than about 2 kDa.

28. The hydrogel system of any one of the preceding claims, wherein the molecular weight of the payload is at least about 1, 2, 4, 10, 15, 30, 50, 70, or 100 kDa.

29. The hydrogel system of any one of the preceding claims, wherein the payload comprises a small molecule encapsulated in another delivery system.

30. The hydrogel system of any one of the preceding claims, wherein the payload comprises a small molecule encapsulated in a nanoparticle, nanowire, nanotube, liposome, or micelle.

31. The hydrogel system of any one of the preceding claims, wherein the payload comprises a protein, peptide, nucleic acid, antibody, or carbohydrate.

32. The hydrogel system of any one of the preceding claims, wherein the payload comprises a drug or therapeutic.

33. The hydrogel system of any one of the preceding claims, wherein the payload is selected from the group consisting of an extracellular matrix component, a cytokine or a growth factor, an antimicrobial, for example, hyaluronic acid, bovine lactoferrin, Epidermal Growth Factor (EGF), Heparin-binding EGF (HB-EGF), Insulin-like Growth Factor (IGF-1), Epiregulin, Platelet-derived growth factor α and β (PDGF-α/β), Transforming growth factor α (TGF-α), Transforming growth factor β (TGF-β), Keratinocyte growth factor (KGF), Hepatocyte growth factor (HGF), or Fibroblast Growth Factor (FGF).

34. The hydrogel system of any one of the preceding claims, wherein the hydrogel is degradable by an enzyme.

35. The hydrogel system of claim 34, wherein the enzyme is an enzyme present at a wound site.

36. The hydrogel system of claim 34, wherein the enzyme is an enzyme that is upregulated at a wound site.

37. The hydrogel system of claim 34, wherein the enzyme is an enzyme added to the hydrogel externally.

38. The hydrogel system of claim 37, wherein the enzyme is added after the hydrogel has been administered to a wound.

39. The hydrogel system of any one of claims 34-38, wherein the enzyme is an extracellular matrix-degrading enzyme.

40. The hydrogel system of any one of claims 34-39, wherein the enzyme is a matrix metalloproteinase.

41. The hydrogel system of any one of claims 34-40, wherein the enzyme is selected from the group consisting of MMP-2, MMP-8, and MMP-9.

42. The hydrogel system of any one of claims 34-41, wherein the enzyme is MMP-8.

43. The hydrogel system of any one of claims 34-42, wherein the enzyme is MMP-9.

44. The hydrogel system of any one of the preceding claims, wherein the system is tuneable based on one or more of GelMA density, methacrylation degree, crosslinking degree, or sequential crosslinking steps, for compatibility with payloads of different sizes and/or release rates.

45. The hydrogel system of any one of the preceding claims, wherein the hydrogel is dried and stored prior to use.

46. The hydrogel system of any one of the preceding claims, wherein the hydrogel is frozen and stored prior to use.

47. The hydrogel system of any one of claims 1-44, wherein the hydrogel is lyophilized and subsequently reconstituted prior to use.

48. The hydrogel system of claim 47, wherein the hydrogel is lyophilized after encapsulation of the payload.

49. The hydrogel system of claim 47, wherein the hydrogel is lyophilized prior to encapsulation of the payload.

50. The hydrogel system of any of the preceding claims, wherein delivering comprises sustained release of the payload.

51. The hydrogel system of claim 50, wherein the sustained release period is over hours, days, weeks, or months.

52. The hydrogel system of claim 50, wherein the sustained release period is between about 12-24 hours, between about 1-2 days, between about 1-5 days, between about 1-14 days, between about 4-10 days, between about 7-10 days, between about 7-14 days, or between about 1-30 days, or about 1 day, about 2 days, about 3 days, about 4 days, about 5 days, about 6 days, about 7 days, about 10 days, about 14 days, about 21 days, or about 28 days.

53. The hydrogel system of claim 50, wherein the sustained release period is between about 1-12 months, such as, between about 1-3 months, about 1-6 months, about 1-9 months, about 3-6 months, about 3-9 months, about 9-12 months, about 6-12 months, or about 1, about 2, about 3, about 5, about 7, about 9, or about 12 months.

54. An enzyme-degradable hydrogel system for delivering a payload to a wound site, the system comprising:
a hydrogel comprising a crosslinkable polymer, such as a chemically-modified biopolymer, for example, chemically-modified gelatin, the hydrogel formed by a method comprising sequential physical and chemical crosslinking steps; and
a payload.

55. A device for delivering a payload, the device comprising:
the hydrogel system according to any one of claims 1-54.

56. The device according to claim 55, wherein the device is a lens, such as a contact lens, an implant, such as a corneal implant, an insert, such as an ocular insert, a patch, a bandage, or a wound dressing.

57. The device according to claim 55 or 56, wherein the device is configured to release the payload over a release period.

58. The device of claim 57, wherein the release period is about 1 to about 5 days, for example, between about 12-24 hours, between about 1-2 days, between about 1-5 days, between about 1-14 days, between about 4-10 days, between about 7-10 days, between about 7-14 days, or between about 1-30 days, or about 1 day, about 2 days, about 3 days, about 4 days, about 5 days, about 6 days, about 7 days, about 10 days, about 14 days, about 21 days, or about 28 days.

59. A method of making a hydrogel system for delivering a payload, the method comprising:
providing a crosslinkable polymer, such as a chemically-modified biopolymer, for example, chemically-modified gelatin;
physical crosslinking of the crosslinkable polymer;
chemical crosslinking of the crosslinkable polymer; and
introducing a payload into the hydrogel so-formed.

60. The method of claim 59, wherein the payload is introduced into the hydrogel prior to physical crosslinking.

61. The method of claim 59, wherein the payload is introduced into the hydrogel prior to chemical crosslinking.

62. The method of claim 59, wherein the payload is introduced into the hydrogel after physical and chemical crosslinking.

63. The method of any one of claims 59-62, wherein the chemically-modified gelatin comprises methacrylated gelatin, acrylated gelatin, thiolated gelatin or a combination thereof.

64. The method of any one of claims 59-63, wherein the chemically-modified gelatin comprises methacrylated gelatin.

65. The method of any one of claims 59-64, wherein physical crosslinking comprises incubating a solution comprising the crosslinkable polymer under suitable conditions and for a sufficient period of time to permit physical crosslinking of at least a portion of the crosslinkable polymer.

66. The method of claim 65, wherein the solution comprises between about 1%-35% (w/v) of the crosslinkable polymer in a suitable diluent, e.g., about 1%-about 5%, about 1%-about 10%, about 5%-about 30%, about 10%-about 35%, about 10%-about 30%, about 10%-about 20%, about 20%-about 30%, about 25%-about 35% or about 1%, about 5%, about 10%, about 15%, about 20%, about 25%, about 30%, or about 35%.

67. The method of claim 66, wherein the solution is an aqueous solution.

68. The method of claim 66 or 67, wherein the diluent is phosphate buffered saline (PBS).

69. The method of any one of claims 59-68, wherein the incubation period is at least 1 hour.

70. The method of any one of claims 59-69, wherein the incubation period is between about 15 minutes to about 3 hours, between about 30 minutes to about 2 hours, between about 45 minutes to about 1.5 hours, or about 15 minutes, about 30 minutes, about 45 minute, about 1 hour, about 1.5 hours, about 2 hours, or about 3 hours.

71. The method of any one of claims 59-70, wherein the physical crosslinking step comprises incubation at a temperature between about 1-about 16° C., between about 2-about 15° C., between about 2-about 10° C., between about 3-about 8° C., between about 4-about 6° C., between about 3-about 5° C. or about 2° C., about 3° C., about 4° C., about 5° C., about 6° C., about 7° C., about 8° C., about 10° C., about 12° C., about 14° C., or about 16° C.

72. The method of any one of claims 59-71, wherein the chemical crosslinking step is a thermal process.

73. The method of any one of claims 59-72, wherein the chemical crosslinking step comprises UV irradiation.

74. The method of claim 73, wherein the UV irradiation takes place in the presence of a photoinitiator.

75. The method of claim 73, wherein photoinitiator is selected from the group consisting of 2-hydroxy-4′-(2-hydroxyethoxy)-2-methylpropiophenone (Irgacure 2959, or IC2959); lithium phenyl-2,4,6-trimethylbenzoylphosphinate (LAP); 2,2′-azobis[2-methyl-n-(2-hydroxyethyl)propionamide] (VA-086); or 2′,4′,5′,7′-tetrabromofluorescein disodium salt (Eosin Y).

76. The method of claim 73, wherein the chemical crosslinking step comprises UV irradiation for between about 10 seconds to about 30 minutes, for example between about 10 seconds to about 30 seconds, between about 30 seconds to about 90 seconds, between about 10 seconds to about 1 minute, between about 1 minute to about 5 minutes, between about 1 minute to about 2 minutes, between about 2 minutes to about 5 minutes, between about 5 minutes to about 10 minutes, between about 10 minutes to about 20 minutes, or about 10 seconds, 30 seconds, 60 seconds, 90 seconds, 2 minutes, 3 minutes, 4 minutes, 5 minutes, 10 minutes, 15 minutes, 20 minutes, or 30 minutes.

77. The method of claim 73, wherein the chemical cross-linking step comprises UV irradiation with between about 360-480 nm, such as between about 360-450 nm, between about 380-480 nm, between about 400-450 nm, between about 360-400 nm, or about 360 nm, 370 nm, 380 nm, 390 nm, 400 nm, 410 nm, 420 nm, 430 nm, 440 nm, 450 nm, 460 nm, 470 nm, or 480 nm.

78. The method of any one of claims 59-77, wherein the chemically-modified gelatin consists of gelatin methacrylate.

79. The method of any one of claims 59-78, wherein gelatin methacrylate is the sole polymer forming the matrix of the hydrogel.

80. The method of any one of claims 59-78, wherein the hydrogel comprises one or more additional polymers.

81. The method of claim 80, wherein the one or more additional polymers is selected from hydrogel polymers, carboxybetaine methacrylate (CBMA), Alginate hydrogel, poly(hydroxylethylmethacrylate) (HEMA), Collagen derivatives, Poly lactic glycolic acid (PLGA), or Acrylamide gels.

82. The method of any one of claims 59-81, wherein at least a portion of the payload is encapsulated within a matrix formed by the hydrogel.

83. The method of claim 82, wherein at least about 50-99% of the payload is encapsulated within a matrix formed by the hydrogel, for example, at least about 50%, 60%, 70%, 75%, 80%, 85%, 90%, 95%, 97%, 98%, or 99%.

84. The method of any one of claims 59-83, wherein at least a portion of the payload is associated with a surface of the hydrogel.

85. The method of any one of claims 59-84, wherein the payload is a drug, marker, cell, or the aforementioned members encapsulated in another delivery vehicle such as a nanoparticle or liposome.

86. The method of any one of claims 59-85, wherein the payload is a small molecule or biologic molecule.

87. The method of any one of claims 59-86, wherein the payload is sufficiently large such that all or a portion of the payload is retained within a matrix formed by the hydrogel until degradation of the hydrogel occurs.

88. The method of any one of claims 59-87, wherein the molecular weight of the payload is less than about 100 kDa, less than about 70 kDa, less than about 50 kDa, less than about 30 kDa, less than about 15 kDa, less than about 10 kDa, less than about 4 kDa, or less than about 2 kDa.

89. The method of any one of claims 59-88, wherein the molecular weight of the payload is at least about 1 kDa.

90. The method of any one of claims 59-89, wherein the payload comprises a small molecule encapsulated in another delivery system.

91. The method of any one of claims 59-90, wherein the payload comprises a small molecule encapsulated in a nanoparticle, nanowire, nanotube, liposome, or micelle.

92. The method of any one of claims 59-91, wherein the payload comprises a protein, peptide, antibody, or carbohydrate.

93. The method of any one of claims 59-92, wherein the payload comprises a drug or therapeutic.

94. The method of any one of claims 59-93, wherein the payload is selected from the group consisting of hyaluronic acid, bovine lactoferrin, Epidermal Growth Factor (EGF), Heparin-binding EGF (HB-EGF), Insulin-like Growth Factor (IGF-1), Epiregulin, Platelet-derived growth factor α and β (PDGF-α/β), Transforming growth factor α (TGF-α), Transforming growth factor β (TGF-β), Keratinocyte growth factor (KGF), Hepatocyte growth factor (HGF), or Fibroblast Growth Factor (FGF)

95. The method of any one of claims 59-94, wherein the hydrogel is degradable by an enzyme.

96. The method of claim 95, wherein the enzyme is an enzyme present at a wound site.

97. The method of claim 95, wherein the enzyme is an enzyme that is upregulated at a wound site.

98. The method of claim 95, wherein the enzyme is an enzyme added to the hydrogel externally.

99. The method of claim 98, wherein the enzyme is added after the hydrogel has been administered to a wound.

100. The method of any one of claims 95-99, wherein the enzyme is an extracellular matrix-degrading enzyme.

101. The method of any one of claims 95-100, wherein the enzyme is a matrix metalloproteinase.

102. The method of any one of claims 95-101, wherein the enzyme is selected from the group consisting of MMP-2, MMP-8, and MMP-9.

103. The method of any one of claims 95-102, wherein the enzyme is MMP-8.

104. The method of any one of claims 95-103, wherein the enzyme is MMP-9.

105. The method of any one of claims 59-104, wherein the system is tuneable based on one or more of GelMA density, methacrylation degree, crosslinking degree, or sequential crosslinking steps, for compatibility with payloads of different sizes and/or release rates.

106. The method of any one of claims 59-105, wherein the method further comprises drying the hydrogel.

107. The method of any one of claims 59-106, wherein the method further comprises freezing the hydrogel.

108. The method of any one of claims 59-105, wherein the method further comprises lyophilizing the hydrogel.

109. The method of claim 108, wherein the hydrogel is lyophilized after encapsulation of the payload.

110. The method of claim 108, wherein the hydrogel is lyophilized prior to encapsulation of the payload.

111. The method of any one of claims 59-110, wherein physical crosslinking comprises incubating the crosslinkable polymer between glass slides. The method of any one of claims 59-110, wherein air is removed from the crosslinkable polymer prior to physical and chemical crosslinking.

112. A method of making a device for delivering a payload, the method comprising, incorporating the hydrogel system according to any one of claims 1-54 into the device.

113. The method according to claim 112, wherein the device is a lens, a contact lens, an implant, a corneal implant, a patch, a bandage, or a wound dressing.

114. A hydrogel formed by the method of any one of claims 59-111.

115. A method of delivering a payload comprising administering the hydrogel system of any one of claims 1-54, the device of any one of claims 55-58 to a patient.

116. A method of treating a wound comprising administering the hydrogel system of any one of claims 1-54, or the device of any one of claims 55-58 to a wound site.

117. The method of claim 116 for treating an ocular wound, a burn wound, a chemical burn wound, an acute wound, a chronic wound, a bone wound, an ulcer, a pressure ulcer, a venous ulcer, or a bedsore.

118. The method of any one of claims 115-117, further comprising administering an enzyme to the hydrogel.

119. The method of any one of claims 115-118, for treating a patient, such as an animal or a human.

120. The method of any one of claims 115-119 for treating an ocular wound, comprising applying an ocular insert comprising the hydrogel system to the lower eyelid pocket.

121. The method of claim 120 wherein an enzyme solution is added in an eye drop to the lower eyelid.

122. The method of any one of claims 115-119 for treating a skin wound, comprising applying a wound dressing comprising the hydrogel system to the wound.

123. The method of claim 122, wherein an enzyme solution is added to the wound dressing.

124. Use of the hydrogel system of any one of claims 1-54, or the device of any one of claims 55-58, for delivering a payload.

125. Use of the hydrogel system of any one of claims 1-54, or the device of any one of claims 55-58, for treating a wound.

126. The use of claim 125 for treating an ocular wound, a burn wound, a chemical burn wound, an acute wound, a chronic wound, a bone wound, an ulcer, a pressure ulcer, a venous ulcer, or a bedsore.

127. The use of any one of claims 124-126, further comprising administering an enzyme to the hydrogel.

128. The use of any one of claims 124-127, for treating a patient, such as an animal or a human.

129. The use of any one of claims 124-128 for treating an ocular wound, comprising applying an ocular insert comprising the hydrogel system to the lower eyelid pocket.

130. The use of claim 129 wherein an enzyme solution is added in an eye drop to the lower eyelid.

131. The use of any one of claims 124-127 for treating a skin wound, comprising applying a wound dressing comprising the hydrogel system to the wound.

132. The use of claim 131, wherein an enzyme solution is added to the wound dressing.

133. A pharmaceutical composition comprising the hydrogel system of any one of claims 1-54 and a pharmaceutically acceptable excipient.

134. The pharmaceutical composition of claim 133, wherein the composition is formulated as a gel or ointment.

135. The pharmaceutical composition of claim 133, wherein the composition is formulated as a patch, implant, or bandage.

136. The pharmaceutical composition of claim 133, further comprising an enzyme for degrading the hydrogel system.

137. The pharmaceutical composition of claim 136, wherein the enzyme is an extracellular matrix-degrading enzyme.

138. The pharmaceutical composition of claim 136 or 137, wherein the enzyme is a matrix metalloproteinase.

139. The pharmaceutical composition of any one of claims 136-138, wherein the enzyme is selected from the group consisting of MMP-2, MMP-8, and MMP-9.

140. The pharmaceutical composition of any one of claims 136-139, wherein the enzyme is MMP-8.

141. The pharmaceutical composition of any one of claims 136-139, wherein the enzyme is MMP-9.

142. Use of the pharmaceutical composition of any one of claims 133-141, for treating a wound.
